SEQUENCE LISTING (3 sequences)

SEQ ID NO: 1 - protein, 14 aa, Artificial Sequence
  MLL peptide
RWRFPARPGTGRRG

SEQ ID NO: 2 - protein, 14 aa, Artificial Sequence
  MLL peptide
RWRFPARPGTGRRG

SEQ ID NO: 3 - protein, 20 aa, Artificial Sequence
  MLL peptide
MAHSCAWAFPGSGSCAWAFP

(19) United States
(12) Patent Application Publication
(10) Pub. No.: US 2017/0119769 A1
(43) Pub. Date: May 4, 2017

(54) SCAFFOLDS FOR INHIBITORS OF MENIN-MLL INTERACTIONS

(71) Applicant: THE TRUSTEES OF THE UNIVERSITY OF PENNSYLVANIA, Philadelphia, PA (US)

(72) Inventors: Xianxin HUA, Winnewood, PA (US); Abdul Bari MUHAMMAD, Strafford, PA (US)

(73) Assignee: THE TRUSTEES OF THE UNIVERSITY OF PENNSYLVANIA, Philadelphia, PA (US)

(21) Appl. No.: 15/317,078

(22) PCT Filed: Jun. 10, 2015
(86) PCT No.: PCT/US15/35091

§ 371 Date: Dec. 7, 2016

Related U.S. Application Data
(60) Provisional application No. 62/010,232, filed on Jun. 10, 2014.

(51) Int. Cl. A61K 31/519; G01N 33/50; A61K 31/445; A61K 31/136; A61K 31/496

(52) U.S. Cl. CPC A61K 31/519; A61K 31/136; A61K 31/496; A61K 31/445; G01N 33/5011; G01N 33/507

(57) ABSTRACT

Disclosed herein are methods and compositions for treating, as well as identifying potential therapeutics for, cancers or diabetes, with compounds that inhibit the activity of menin, preferably the compounds are capable of inhibiting MLL binding to menin.

### CROSS-REFERENCE TO RELATED APPLICATIONS

This application claims the benefit of priority from U.S. Provisional Patent Application No. 62/010,232, filed Jun. 10, 2014, which is incorporated herein by reference in its entirety.

### GOVERNMENT INTEREST STATEMENT

This invention was made with government support under Grant Number R01-DK085121 02, awarded by the National Institutes of Health. The government has certain rights in the invention.

### FIELD OF THE INVENTION

This invention relates to compounds and methods for treating cancers or diabetes or for inhibiting the growth or proliferation of cancer cells or increasing the growth or proliferation of beta cells, with compounds that inhibit the activity of menin, preferably the compounds are capable of inhibiting MLL binding to menin.

### BACKGROUND OF THE INVENTION

Multiple endocrine neoplasia type 1 (MEN1) is a dominantly inherited tumor syndrome that results from the mutation of the tumor suppressor gene Men1, which encodes menin Menin interacts with multiple proteins that play critical roles in the regulation of cell proliferation, including JunD, Smad 3, and activator of S-phase kinase. Activator of S-phase kinase is the regulatory factor for protein kinase cdc7 that is required for initiation of DNA replication and menin functionally represses the activity of activator of S-phase kinase. In addition, menin interacts with a protein complex containing the mixed lineage leukemia protein and up-regulates transcription of various target genes, including the cyclin-dependent kinase (CDK) inhibitors p27^{Kip1} and p^{18Ink4c}, in transformed fibroblasts and insulinoma cells. Tumors derived from mice heterozygous for Men1 display loss of heterozygosity, confirming the role of menin as a tumor suppressor. Tumors arise in the parathyroid, pituitary, and pancreatic islet cells from the mice in which Men1 is conditionally inactivated in these respective organs, establishing an important role for menin in suppressing tumor development in endocrine organs.

The Mixed Lineage Leukemia (Mll) gene forms chromosomal translocations with multiple gene partners, leading to the expression of MLL fusion proteins (MLL-FPs), which cause acute leukemias (such as acute myeloid leukemia and acute lymphoblastic leukemia) with particularly poor prognoses. The nuclear DNA binding protein menin is necessary for the recruitment of MLL-FPs and wild-type (WT) MLL to target genes, including Hox genes. Both MLL-FPs and WT MLL are necessary to maintain MLL-AF9-mediated leukemogenesis, highlighting menin as a central node in controlling two distinct mediators of leukemogenesis. The region of MLL that interacts with menin has been mapped to a small N-terminal portion of the protein, which is retained in MLL-FPs, presenting a potential target that could inhibit menin's interaction with both MLL-FPs and WT MLL to treat this disease (Huang et al., (2012) Nature 482:542). Compounds with nanomolar-affinity have been identified, which inhibit the interaction between menin and MLL and reverse the oncogenic activity of MLL-AF9 cells (Grembecka et al., (2012) Nat Chem Biol. 8:277).

Diabetes mellitus (DM) describes several syndromes of abnormal carbohydrate metabolism, characterized by hyperglycemia. It is associated with a relative or absolute impairment in insulin secretion, along with varying degrees of peripheral resistance to the action of insulin. The chronic hyperglycemia of diabetes is associated with long-term damage, dysfunction, and failure of various organs, especially the eyes, kidneys, nerves, heart, and blood vessels.

There are two major forms of diabetes: Type I diabetes, also referred to as insulin-dependent diabetes; and Type II diabetes, also referred to as noninsulin dependent diabetes. When inadequate amounts of insulin are present to compensate for insulin resistance and adequately control glucose, a state of impaired glucose tolerance develops. In a significant number of individuals, the plasma glucose level rises, resulting in the clinical state of diabetes. Insulin stimulates glucose uptake by skeletal muscle and adipose tissues primarily through translocation of the glucose transporter 4 from the intracellular storage sites of the cell surface.

Diabetes is often associated with high fat diet and obesity. The majority of diabetic patients are treated either with hypoglycemic agents which act by stimulating release of insulin from beta cells, or with agents that enhance the tissue sensitivity of the patients towards insulin, or with insulin. Increased islet proliferation as an acute consequence of Men1 deletion also suggests that inhibition of Men1 expression or function can be employed as a means to specifically stimulate proliferation of islet cells, over 80% of which are insulin-secreting beta cells, to treat diabetes.

There is a need in the art for compositions and methods that inhibit the activity of or bind to menin. There is also a need in the art for compositions and methods for treating cancers or diabetes, with compounds that inhibit the activity of menin, preferably the compounds are capable of inhibiting MLL binding to menin There is also a need in the art for compositions and methods for inhibiting the growth or proliferation of cancer or precancerous cells, with compounds that inhibit the activity of menin, preferably the compounds are capable of inhibiting MLL binding to menin There is also a need in the art for compositions and methods for increasing the growth or proliferation of beta cells, with compounds that inhibit the activity of menin There is also a need in the art for compositions and methods for identifying potential cancer or diabetes therapeutics with compounds that inhibit the activity of menin, preferably the compounds are capable of inhibiting MLL binding to menin.

### SUMMARY OF THE INVENTION

In one aspect, methods for treating diabetes in a subject are provided, the methods include: the step of administering to a subject with diabetes a therapeutically effective amount of a pharmaceutical composition of a compound of Formula B or a pharmaceutically acceptable salt, solvate, hydrate or stereoisomer thereof:

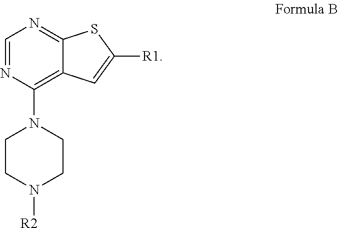

R1 and R2 may comprise any substituents which result in compounds which inhibit the interaction of MLL proteins with menin.

In another aspect, methods for enhancing pancreatic β-cell proliferation in a subject are provided, the methods include: the step of administering to a subject with diabetes a therapeutically effective amount of a pharmaceutical composition of a compound of Formula B or a pharmaceutically acceptable salt, solvate, hydrate or stereoisomer thereof.

In one aspect, methods for treating cancer in a subject are provided, the methods include:

the step of administering to a subject with cancer a therapeutically effective amount of a pharmaceutical composition of a compound of Formula A or a pharmaceutically acceptable salt, solvate, hydrate or stereoisomer thereof:

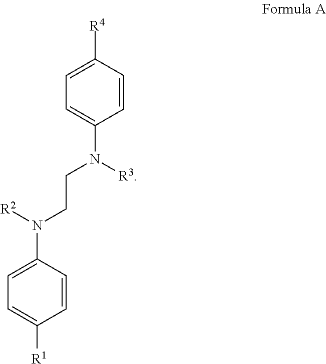

wherein R¹ and R⁴ are each independently selected from the group consisting of hydrogen, halogen, NR^{a}R^{b}, hydroxyl, linear or branched C₁-C₆ alkyl, linear or branched C₂-C₆ alkenyl, linear or branched C₂-C₆ alkynyl, C₃-C₈ cycloalkyl, C₃-C₈ cycloalkenyl, and C₁-C₆alkoxy;
   wherein said alkyls, alkenyls, alkynyls, cycloalkyls, cycloalkenyls and alkoxys of R¹ and R⁴ are unsubstituted or substituted with at least one halogen;
   wherein R^{a} and R^{b} are each independently selected from group consisting of hydrogen, linear or branched C₁-C₄ alkyl, linear or branched C₂-C₄ alkenyl, C₂-C₄ alkynyl, C₃-C₆ cycloalkyl, and C₃-C₆ cycloalkenyl;
   wherein said alkyls, alkenyls, alkynyls, cycloalkyls, cycloalkenyls of R^{a} and R^{b} are unsubstituted or substituted with at least one halogen;
wherein R² and R³ are each independently selected from group consisting of hydrogen, linear or branched C₁-C₆ alkyl, linear or branched C₂-C₆ alkenyl, linear or branched C₂-C₆ alkynyl, C₃-C₈ cycloalkyl, and C₃-C₈ cycloalkenyl;
   wherein said alkyls, alkenyls, alkynyls, cycloalkyls, cycloalkenyls of R² and R³ are unsubstituted or substituted with at least one halogen.

In another aspect, methods for treating cancer in a subject are provided, the methods include: the step of administering to a subject with cancer a therapeutically effective amount of a pharmaceutical composition of Compound III or a pharmaceutically acceptable salt, solvate, hydrate or stereoisomer thereof:

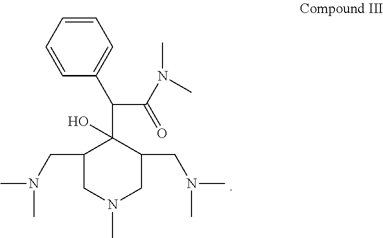

In a further aspect, methods for treating cancer in a subject are provided, the methods include: the step of administering to a subject with cancer a therapeutically effective amount of a pharmaceutical composition of Compound IV or a pharmaceutically acceptable salt, solvate, hydrate or stereoisomer thereof:

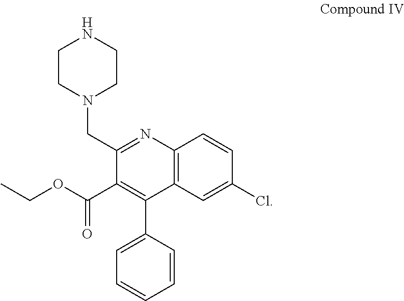 [SMILES: CCOC(=O)c1c(CN2CCNCC2)nc2ccc(Cl)cc2c1-c1ccccc1]

In an additional aspect, methods for treating diabetes in a subject are provided, the methods include: the step of administering to a subject with diabetes a therapeutically effective amount of a pharmaceutical composition of a compound of Formula A or a pharmaceutically acceptable salt, solvate, hydrate or stereoisomer thereof:

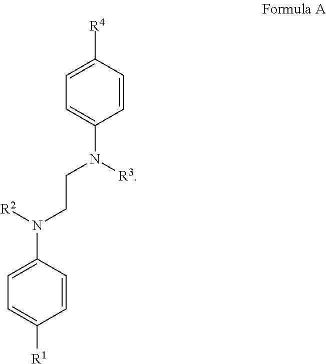

wherein R¹ and R⁴ are each independently selected from the group consisting of hydrogen, halogen, NR^{a}R^{b}, hydroxyl, linear or branched C₁-C₆ alkyl, linear or branched C₂-C₆ alkenyl, linear or branched C₂-C₆ alkynyl, C₃-C₈ cycloalkyl, C₃-C₈ cycloalkenyl, and C₁-C₆alkoxy;
   wherein said alkyls, alkenyls, alkynyls, cycloalkyls, cycloalkenyls and alkoxys of R¹ and R⁴ are unsubstituted or substituted with at least one halogen;
   wherein R^{a} and R^{b} are each independently selected from group consisting of hydrogen, linear or branched C₁-C₄ alkyl, linear or branched C₂-C₄ alkenyl, C₂-C₄ alkynyl, C₃-C₆ cycloalkyl, and C₃-C₆ cycloalkenyl;
wherein said alkyls, alkenyls, alkynyls, cycloalkyls, cycloalkenyls of R^{a} and R^{b} are unsubstituted or substituted with at least one halogen;
wherein R² and R³ are each independently selected from group consisting of hydrogen, linear or branched C₁-C₆ alkyl, linear or branched C₂-C₆ alkenyl, linear or branched C₂-C₆ alkynyl, C₃-C₈ cycloalkyl, and C₃-C₈ cycloalkenyl;
   wherein said alkyls, alkenyls, alkynyls, cycloalkyls, cycloalkenyls of R² and R³ are unsubstituted or substituted with at least one halogen.

In an other aspect, methods for treating diabetes in a subject are provided, the methods include: the step of administering to a subject with diabetes a therapeutically effective amount of a pharmaceutical composition of Compound III or a pharmaceutically acceptable salt, solvate, hydrate or stereoisomer thereof:

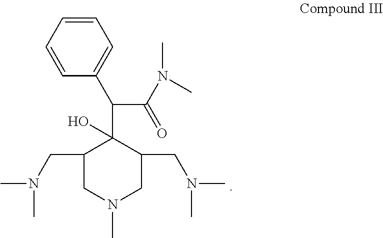

In yet another aspect, methods for treating diabetes in a subject are provided, the methods include: the step of administering to a subject with diabetes a therapeutically effective amount of a pharmaceutical composition of Compound IV or a pharmaceutically acceptable salt, solvate, hydrate or stereoisomer thereof:

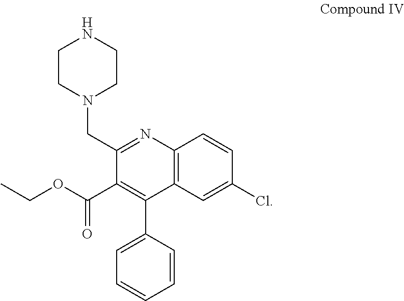 [SMILES: CCOC(=O)c1c(CN2CCNCC2)nc2ccc(Cl)cc2c1-c1ccccc1]

In yet another aspect, methods for inhibiting the growth or proliferation of a cancerous or precancerous cell are provided, the methods include: the step contacting a cancer or precancerous cell with an effective amount of a pharmaceutical composition of a compound of Formula A or a pharmaceutically acceptable salt, solvate, hydrate or stereoisomer thereof:

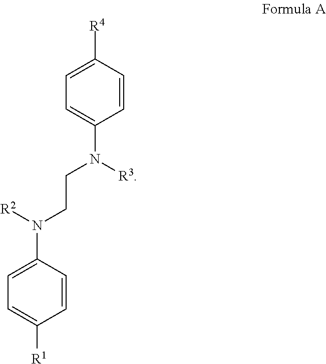

wherein R¹ and R⁴ are each independently selected from the group consisting of hydrogen, halogen, NR^{a}R^{b}, hydroxyl, linear or branched C₁-C₆ alkyl, linear or branched C₂-C₆ alkenyl, linear or branched C₂-C₆ alkynyl, C₃-C₈ cycloalkyl, C₃-C₈ cycloalkenyl, and C₁-C₆alkoxy;
   wherein said alkyls, alkenyls, alkynyls, cycloalkyls, cycloalkenyls and alkoxys of R¹ and R⁴ are unsubstituted or substituted with at least one halogen;
   wherein R^{a} and R^{b} are each independently selected from group consisting of hydrogen, linear or branched C₁-C₄ alkyl, linear or branched C₂-C₄ alkenyl, C₂-C₄ alkynyl, C₃-C₆ cycloalkyl, and C₃-C₆ cycloalkenyl;
   wherein said alkyls, alkenyls, alkynyls, cycloalkyls, cycloalkenyls of R^{a} and R^{b} are unsubstituted or substituted with at least one halogen;
wherein R² and R³ are each independently selected from group consisting of hydrogen, linear or branched C₁-C₆ alkyl, linear or branched C₂-C₆ alkenyl, linear or branched C₂-C₆ alkynyl, C₃-C₈ cycloalkyl, and C₃-C₈ cycloalkenyl;
   wherein said alkyls, alkenyls, alkynyls, cycloalkyls, cycloalkenyls of R² and R³ are unsubstituted or substituted with at least one halogen.

In yet an additional aspect, methods for inhibiting the growth or proliferation of a cancerous or precancerous cell are provided, the methods include: the step contacting a cancer or precancerous cell with an effective amount of a pharmaceutical composition of Compound III or a pharmaceutically acceptable salt, solvate, hydrate or stereoisomer thereof:

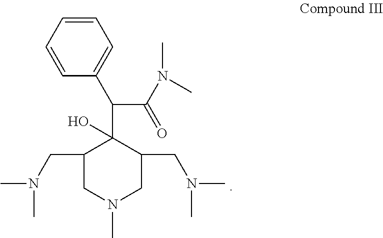

In yet an other aspect, methods for inhibiting the growth or proliferation of a cancerous or precancerous cell are provided, the methods include: the step contacting a cancer or precancerous cell with an effective amount of a pharmaceutical composition of Compound IV or a pharmaceutically acceptable salt, solvate, hydrate or stereoisomer thereof:

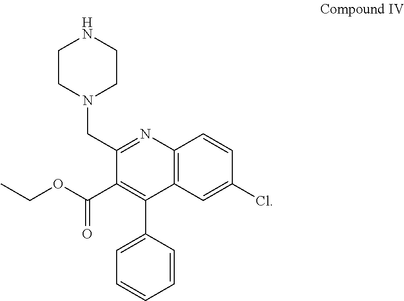 [SMILES: CCOC(=O)c1c(CN2CCNCC2)nc2ccc(Cl)cc2c1-c1ccccc1]

In a further aspect, methods for screening a potential cancer therapeutic are provided, the methods include the steps of: growing cells containing a chromosomal translocation of the Mixed Lineage Leukemia (Mll) gene in the presence of a compound suspected of being a cancer therapeutic, growing said cells in the presence of a reference compound, determining the rate of growth of said cells in the presence of said compound and the rate of growth of said cells in the presence of a reference compound and comparing the growth rate of said cells, wherein a slower rate of growth of said cells in the presence of said compound than in the presence of said reference compound is indicative of a cancer therapeutic, wherein said reference compound is a compound of Formula A or a pharmaceutically acceptable salt, solvate, hydrate or stereoisomer thereof:

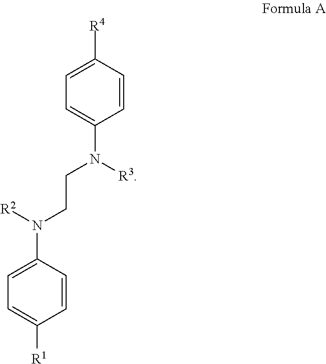

wherein R¹ and R⁴ are each independently selected from the group consisting of hydrogen, halogen, NR^{a}R^{b}, hydroxyl, linear or branched C₁-C₆ alkyl, linear or branched C₂-C₆ alkenyl, linear or branched C₂-C₆ alkynyl, C₃-C₈ cycloalkyl, C₃-C₈ cycloalkenyl, and C₁-C₆alkoxy;
   wherein said alkyls, alkenyls, alkynyls, cycloalkyls, cycloalkenyls and alkoxys of R¹ and R⁴ are unsubstituted or substituted with at least one halogen;
   wherein R^{a} and R^{b} are each independently selected from group consisting of hydrogen, linear or branched C₁-C₄ alkyl, linear or branched C₂-C₄ alkenyl, C₂-C₄ alkynyl, C₃-C₆ cycloalkyl, and C₃-C₆ cycloalkenyl;
   wherein said alkyls, alkenyls, alkynyls, cycloalkyls, cycloalkenyls of R^{a} and R^{b} are unsubstituted or substituted with at least one halogen;
wherein R² and R³ are each independently selected from group consisting of hydrogen, linear or branched C₁-C₆ alkyl, linear or branched C₂-C₆ alkenyl, linear or branched C₂-C₆ alkynyl, C₃-C₈ cycloalkyl, and C₃-C₈ cycloalkenyl;
   wherein said alkyls, alkenyls, alkynyls, cycloalkyls, cycloalkenyls of R² and R³ are unsubstituted or substituted with at least one halogen.

In an additional aspect, methods for screening a potential cancer therapeutic are provided, the methods include the steps of: growing cells containing a chromosomal translocation of the Mixed Lineage Leukemia (Mll) gene in the presence of a compound suspected of being a cancer therapeutic, growing said cells in the presence of a reference compound, determining the rate of growth of said cells in the presence of said compound and the rate of growth of said cell in the presence of a reference compound and comparing the growth rate of said cells, wherein a slower rate of growth of said cells in the presence of said compound than in the presence of said reference compound is indicative of a cancer therapeutic, wherein said reference compound is Compound III or a pharmaceutically acceptable salt, solvate, hydrate or stereoisomer thereof:

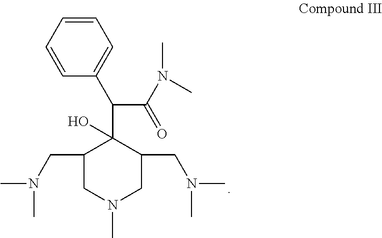

In an other aspect, methods for screening a potential cancer therapeutic are provided, the methods include the steps of: growing cells containing a chromosomal translocation of the Mixed Lineage Leukemia (Mll) gene in the presence of a compound suspected of being a cancer therapeutic, growing said cells in the presence of a reference compound, determining the rate of growth of said cells in the presence of said compound and the rate of growth of said cells in the presence of a reference compound and comparing the growth rate of said cells, wherein a slower rate of growth of said cells in the presence of said compound than in the presence of said reference compound is indicative of a cancer therapeutic, wherein said reference compound is Compound IV or a pharmaceutically acceptable salt, solvate, hydrate or stereoisomer thereof:

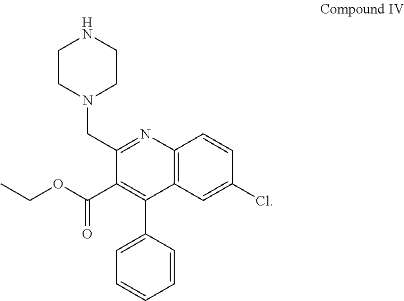 [SMILES: CCOC(=O)c1c(CN2CCNCC2)nc2ccc(Cl)cc2c1-c1ccccc1]

In another aspect, methods of screening a potential diabetes therapeutic are provided, the methods include the steps of: growing pancreatic β-cells in the presence of a compound suspected of being a diabetes therapeutic, growing pancreatic β-cells in the presence of a reference compound, determining the rate of growth of the pancreatic β-cells in the presence of said compound and the rate of growth of the pancreatic β-cells in the presence of the reference compound and comparing the growth rate of the pancreatic β-cells, wherein a higher rate of growth of pancreatic β-cells in the presence of said compound than in the presence of said reference compound is indicative of a diabetes therapeutic, wherein said reference compound is a compound of Formula B or a pharmaceutically acceptable salt, solvate, hydrate or stereoisomer thereof.

In another aspect, methods of identifying a compound that enhances pancreatic β-cell proliferation are provided, the methods include the steps of: growing β-cells in the presence of a compound suspected of enhancing pancreatic β-cell proliferation, growing pancreatic β-cells in the presence of a reference compound, determining the rate of growth of the pancreatic β-cells in the presence of said compound and the rate of growth of the pancreatic β-cells in the presence of the reference compound and comparing the growth rate of the pancreatic β-cells, wherein a higher rate of growth of pancreatic β-cells in the presence of said compound than in the presence of said reference compound is indicative of a compound that enhances pancreatic β-cell proliferation, wherein said reference compound is a compound of Formula B or a pharmaceutically acceptable salt, solvate, hydrate or stereoisomer thereof.

Unless otherwise defined, all technical and scientific terms used herein have the same meaning as commonly understood by one of ordinary skill in the art to which this invention belongs. Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description. It is also contemplated that whenever appropriate, any embodiment of the present invention can be combined with one or more other embodiments of the present invention, even though the embodiments are described under different aspects of the present invention.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts the structures of four compounds from the in silico screening that inhibit the interaction between menin and MLL-peptide;

FIG. 2 is a plot of the normalized inhibition by each of the compounds depicted in FIG. 1 of menin and MLL-peptide binding and each compounds “IC₅₀”, where the IC₅₀ is relative to the inhibition of 1 μM Biotin-MLL-peptide being 100% inhibition;

FIG. 3 plots the expression relative to a (dimethyl sulfoxide) DMSO control of several genes at a concentration of 10 or 50 μM, respectively, of Compound I in AT-1 cells (a mouse MLL-AF9 transformed cell line);

FIG. 4 plots growth as a function of time of THP-1 cells (a human MLL-AF9 transformed cell line) in the presence of 10 or 50 μM, respectively, of Compound I or a control (DMSO);

FIG. 5 plots growth as a function of time of AT-1 cells in the presence of 10 or 50 μM, respectively, of Compound I or a control (DMSO);

FIG. 6 plots growth as a function of time of AT-1 cells in the presence of 10 or 50 μM, respectively, of Compound II or a control (DMSO);

FIG. 7 plots the expression relative to a DMSO control of several genes at a concentration of 50 μM of Compound II in AT-1 cells;

FIG. 8 plots the expression relative to a DMSO control of several genes at a concentration of 10 or 50 μM, respectively, of Compound III in AT-1 cells;

FIG. 9 plots growth as a function of time of AT-1 cells in the presence of 10 or 50 μM, respectively, of Compound III or a control (DMSO);

FIG. 10 plots growth as a function of time of THP-1 cells in the presence of 10 or 50 μM, respectively, of Compound III or a control (DMSO);

FIG. 11 plots the expression relative to a DMSO control of several genes at a concentration of 1 or 2 μM, respectively, of Compound IV in AT-1 cells;

FIG. 12 plots growth as a function of time of AT-1 cells in the presence of several concentrations ranging from 1 to 50 μM of Compound IV or a control (DMSO); and

FIG. 13 plots growth as a function of time of THP-1 cells in the presence of several concentrations ranging from 1 to 50 μM of Compound IV or a control (DMSO).

FIG. 14. Menin inhibitor (MI-2-2) increases proliferation in murine islets, as evidenced by increased number of cells positive for the proliferation marker Ki67. Representative images showing increased Ki67 staining in murine islets treated with 500 nM MI-2-2 for two days (B) compared with vehicle control (A). Isolated murine islets were pre-cultured for one day before treatment with drug or vehicle. Quantification of Ki67 positive cells per unit area shows a dose dependent increase upon treatment with MI-2-2 (C).

FIG. 15. Menin inhibition antagonizes menin mediated suppression of cell proliferation. Menin inhibitor (MI-2-2) increases proliferation of menin-expressing MEF cells, but not menin-null (vector) cells. Vector and menin-expressing MEFs (inset) were seeded at 0.25×10⁶ cells/10 cm dish on day 0, treated with either DMSO or increasing concentrations of MI-2-2 from day 1 as indicated, and counted after six days of treatment.

FIG. 16. Menin inhibition increases expression of the GLP1 receptor Human islets were cultured in a humidified incubator with 5% CO₂ at 37° C. After 24 hours, the islets were treated with vehicle control (DMSO) or varying concentrations of MI-2-2 (50 nM, 200 nM or 500 nM) for 2 days. The islets were harvested and. RNA was isolated and was reverse transcribed to cDNA. cDNA was quantitated by real time quantitative PCR and normalized using β-actin as endogenous control.

### DETAILED DESCRIPTION OF THE PRESENT INVENTION

In one aspect, methods for treating diabetes in a subject are provided, the methods include: the step of administering to a subject with diabetes a therapeutically effective amount of a pharmaceutical composition of a compound of Formula B or a pharmaceutically acceptable salt, solvate, hydrate or stereoisomer thereof:

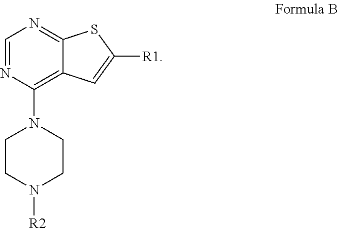

R1 and R2 may comprise any substituents which result in compounds which inhibit the interaction of MLL proteins with menin. In some embodiments, the diabetes being treated is Type I diabetes. In some embodiments, the diabetes being treated is Type II diabetes.

In another aspect, methods for enhancing pancreatic β-cell proliferation in a subject are provided, the methods include: the step of administering to a subject with diabetes a therapeutically effective amount of a pharmaceutical composition of a compound of Formula B or a pharmaceutically acceptable salt, solvate, hydrate or stereoisomer thereof. In some embodiments, the diabetes being treated is Type I diabetes. In some embodiments, the diabetes being treated is Type II diabetes.

In another aspect, methods for treating cancer in a subject are provided, the methods include: the step of administering to a subject with cancer a therapeutically effective amount of a pharmaceutical composition of a compound of Formula A or a pharmaceutically acceptable salt, solvate, hydrate or stereoisomer thereof:

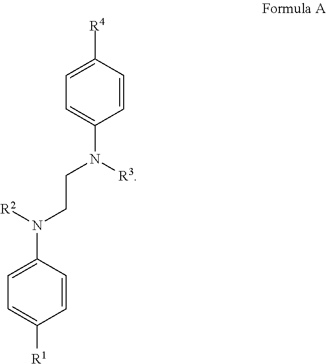

wherein R¹ and R⁴ are each independently selected from the group consisting of hydrogen, halogen, NR^{a}R^{b}, hydroxyl, linear or branched C₁-C₆ alkyl, linear or branched C₂-C₆ alkenyl, linear or branched C₂-C₆ alkynyl, C₃-C₈ cycloalkyl, C₃-C₈ cycloalkenyl, and C₁-C₆alkoxy;
   wherein said alkyls, alkenyls, alkynyls, cycloalkyls, cycloalkenyls and alkoxys of R¹ and R⁴ are unsubstituted or substituted with at least one halogen;
   wherein R^{a} and R^{b} are each independently selected from group consisting of hydrogen, linear or branched C₁-C₄ alkyl, linear or branched C₂-C₄ alkenyl, C₂-C₄ alkynyl, C₃-C₆ cycloalkyl, and C₃-C₆ cycloalkenyl;
   wherein said alkyls, alkenyls, alkynyls, cycloalkyls, cycloalkenyls of R^{a} and R^{b} are unsubstituted or substituted with at least one halogen;
wherein R² and R³ are each independently selected from group consisting of hydrogen, linear or branched C₁-C₆ alkyl, linear or branched C₂-C₆ alkenyl, linear or branched C₂-C₆ alkynyl, C₃-C₈ cycloalkyl, and C₃-C₈ cycloalkenyl;
   wherein said alkyls, alkenyls, alkynyls, cycloalkyls, cycloalkenyls of R² and R³ are unsubstituted or substituted with at least one halogen.

In another aspect, methods for treating cancer in a subject are provided, the methods include: the step of administering to a subject with cancer a therapeutically effective amount of a pharmaceutical composition of Compound III or a pharmaceutically acceptable salt, solvate, hydrate or stereoisomer thereof:

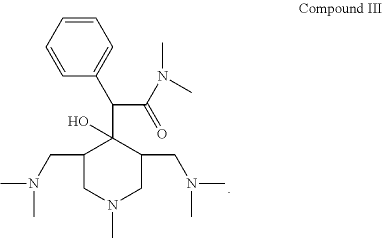

In a further aspect, methods for treating cancer in a subject are provided, the methods include: the step of administering to a subject with cancer a therapeutically effective amount of a pharmaceutical composition of Compound IV or a pharmaceutically acceptable salt, solvate, hydrate or stereoisomer thereof:

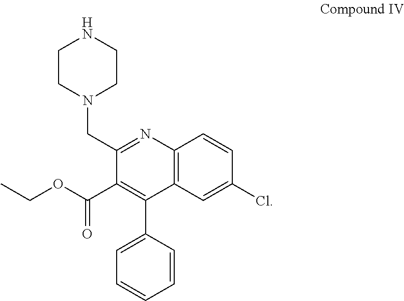 [SMILES: CCOC(=O)c1c(CN2CCNCC2)nc2ccc(Cl)cc2c1-c1ccccc1]

In an additional aspect, methods for treating diabetes in a subject are provided, the methods include: the step of administering to a subject with diabetes a therapeutically effective amount of a pharmaceutical composition of a compound of Formula A or a pharmaceutically acceptable salt, solvate, hydrate or stereoisomer thereof:

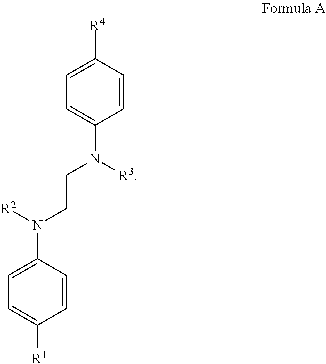

wherein R¹ and R⁴ are each independently selected from the group consisting of hydrogen, halogen, NR^{a}R^{b}, hydroxyl, linear or branched C₁-C₆ alkyl, linear or branched C₂-C₆ alkenyl, linear or branched C₂-C₆ alkynyl, C₃-C₈ cycloalkyl, C₃-C₈ cycloalkenyl, and C₁-C₆alkoxy;
   wherein said alkyls, alkenyls, alkynyls, cycloalkyls, cycloalkenyls and alkoxys of R¹ and R⁴ are unsubstituted or substituted with at least one halogen;
   wherein R^{a} and R^{b} are each independently selected from group consisting of hydrogen, linear or branched C₁-C₄ alkyl, linear or branched C₂-C₄ alkenyl, C₂-C₄ alkynyl, C₃-C₆ cycloalkyl, and C₃-C₆ cycloalkenyl;
   wherein said alkyls, alkenyls, alkynyls, cycloalkyls, cycloalkenyls of R^{a} and R^{b} are unsubstituted or substituted with at least one halogen;
wherein R² and R³ are each independently selected from group consisting of hydrogen, linear or branched C₁-C₆ alkyl, linear or branched C₂-C₆ alkenyl, linear or branched C₂-C₆ alkynyl, C₃-C₈ cycloalkyl, and C₃-C₈ cycloalkenyl;
   wherein said alkyls, alkenyls, alkynyls, cycloalkyls, cycloalkenyls of R² and R³ are unsubstituted or substituted with at least one halogen.

In an other aspect, methods for treating diabetes in a subject are provided, the methods include: the step of administering to a subject with diabetes a therapeutically effective amount of a pharmaceutical composition of Compound III or a pharmaceutically acceptable salt, solvate, hydrate or stereoisomer thereof:

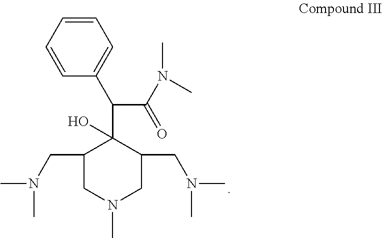

In yet another aspect, methods for treating diabetes in a subject are provided, the methods include: the step of administering to a subject with diabetes a therapeutically effective amount of a pharmaceutical composition of Compound W or a pharmaceutically acceptable salt, solvate, hydrate or stereoisomer thereof:

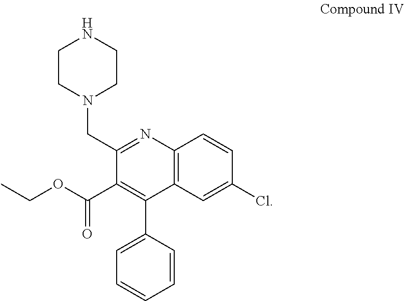 [SMILES: CCOC(=O)c1c(CN2CCNCC2)nc2ccc(Cl)cc2c1-c1ccccc1]

In yet another aspect, methods for inhibiting the growth or proliferation of a cancerous or precancerous cell are provided, the methods include: the step contacting a cancer or precancerous cell with an effective amount of a pharmaceutical composition of a compound of Formula A or a pharmaceutically acceptable salt, solvate, hydrate or stereoisomer thereof:

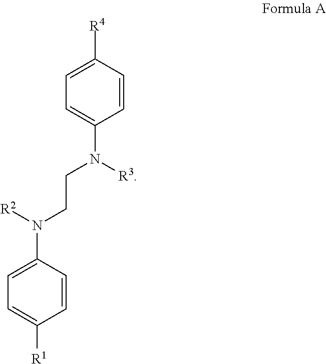

wherein R¹ and R⁴ are each independently selected from the group consisting of hydrogen, halogen, NR^{a}R^{b}, hydroxyl, linear or branched C₁-C₆ alkyl, linear or branched C₂-C₆ alkenyl, linear or branched C₂-C₆ alkynyl, C₃-C₈ cycloalkyl, C₃-C₈ cycloalkenyl, and C₁-C₆alkoxy;
   wherein said alkyls, alkenyls, alkynyls, cycloalkyls, cycloalkenyls and alkoxys of R¹ and R⁴ are unsubstituted or substituted with at least one halogen;
   wherein R^{a} and R^{b} are each independently selected from group consisting of hydrogen, linear or branched C₁-C₄ alkyl, linear or branched C₂-C₄ alkenyl, C₂-C₄ alkynyl, C₃-C₆ cycloalkyl, and C₃-C₆ cycloalkenyl;
   wherein said alkyls, alkenyls, alkynyls, cycloalkyls, cycloalkenyls of R^{a} and R^{b} are unsubstituted or substituted with at least one halogen;
wherein R² and R³ are each independently selected from group consisting of hydrogen, linear or branched C₁-C₆ alkyl, linear or branched C₂-C₆ alkenyl, linear or branched C₂-C₆ alkynyl, C₃-C₈ cycloalkyl, and C₃-C₈ cycloalkenyl;
   wherein said alkyls, alkenyls, alkynyls, cycloalkyls, cycloalkenyls of R² and R³ are unsubstituted or substituted with at least one halogen.

In yet an additional aspect, methods for inhibiting the growth or proliferation of a cancerous or precancerous cell are provided, the methods include: the step contacting a cancer or precancerous cell with an effective amount of a pharmaceutical composition of Compound III or a pharmaceutically acceptable salt, solvate, hydrate or stereoisomer thereof:

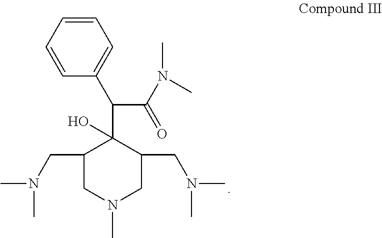

In yet an other aspect, methods for inhibiting the growth or proliferation of a cancerous or precancerous cell are provided, the methods include: the step contacting a cancer or precancerous cell with an effective amount of a pharmaceutical composition of Compound IV or a pharmaceutically acceptable salt, solvate, hydrate or stereoisomer thereof:

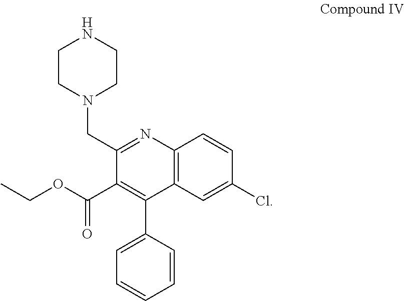 [SMILES: CCOC(=O)c1c(CN2CCNCC2)nc2ccc(Cl)cc2c1-c1ccccc1]

In a further aspect, methods for screening a potential cancer therapeutic are provided, the methods include the steps of: growing cells containing a chromosomal translocation of the Mixed Lineage Leukemia (Mll) gene in the presence of a compound suspected of being a cancer therapeutic, growing said cells in the presence of a reference compound, determining the rate of growth of said cells in the presence of said compound and the rate of growth of said cells in the presence of a reference compound and comparing the growth rate of said cells, wherein a slower rate of growth of said cells in the presence of said compound than in the presence of said reference compound is indicative of a cancer therapeutic, wherein said reference compound is a compound of Formula A or a pharmaceutically acceptable salt, solvate, hydrate or stereoisomer thereof:

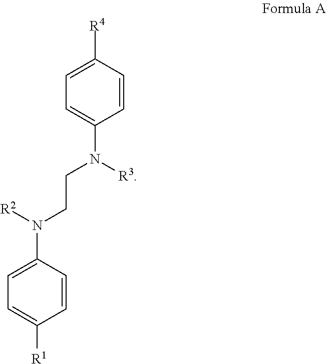

wherein R¹ and R⁴ are each independently selected from the group consisting of hydrogen, halogen, NR^{a}R^{b}, hydroxyl, linear or branched C₁-C₆ alkyl, linear or branched C₂-C₆ alkenyl, linear or branched C₂-C₆ alkynyl, C₃-C₈ cycloalkyl, C₃-C₈ cycloalkenyl, and C₁-C₆alkoxy;
   wherein said alkyls, alkenyls, alkynyls, cycloalkyls, cycloalkenyls and alkoxys of R¹ and R⁴ are unsubstituted or substituted with at least one halogen;
   wherein R^{a} and R^{b} are each independently selected from group consisting of hydrogen, linear or branched C₁-C₄ alkyl, linear or branched C₂-C₄ alkenyl, C₂-C₄ alkynyl, C₃-C₆ cycloalkyl, and C₃-C₆ cycloalkenyl;
   wherein said alkyls, alkenyls, alkynyls, cycloalkyls, cycloalkenyls of R^{a} and R^{b} are unsubstituted or substituted with at least one halogen;
wherein R² and R³ are each independently selected from group consisting of hydrogen, linear or branched C₁-C₆ alkyl, linear or branched C₂-C₆ alkenyl, linear or branched C₂-C₆ alkynyl, C₃-C₈ cycloalkyl, and C₃-C₈ cycloalkenyl;
   wherein said alkyls, alkenyls, alkynyls, cycloalkyls, cycloalkenyls of R² and R³ are unsubstituted or substituted with at least one halogen.

In an additional aspect, methods for screening a potential cancer therapeutic are provided, the methods include the steps of: growing cells containing a chromosomal translocation of the Mixed Lineage Leukemia (Mll) gene in the presence of a compound suspected of being a cancer therapeutic, growing said cells in the presence of a reference compound, determining the rate of growth of said cells in the presence of said compound and the rate of growth of said cells in the presence of a reference compound and comparing the growth rate of said cells, wherein a slower rate of growth of said cells in the presence of said compound than in the presence of said reference compound is indicative of a cancer therapeutic, wherein said reference compound is Compound III or a pharmaceutically acceptable salt, solvate, hydrate or stereoisomer thereof:

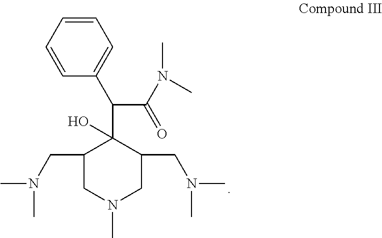

In an other aspect, methods for screening a potential cancer therapeutic are provided, the methods include the steps of: growing cells containing a chromosomal translocation of the Mixed Lineage Leukemia (Mll) gene in the presence of a compound suspected of being a cancer therapeutic, growing said cells in the presence of a reference compound, determining the rate of growth of said cells in the presence of said compound and the rate of growth of said cells in the presence of a reference compound and comparing the growth rate of said cells, wherein a slower rate of growth of said cells in the presence of said compound than in the presence of said reference compound is indicative of a cancer therapeutic, wherein said reference compound is Compound IV or a pharmaceutically acceptable salt, solvate, hydrate or stereoisomer thereof:

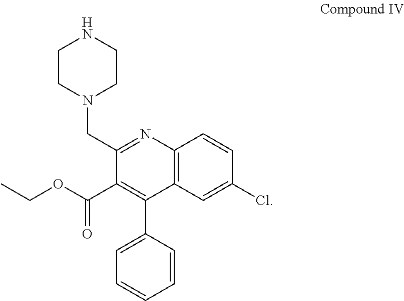 [SMILES: CCOC(=O)c1c(CN2CCNCC2)nc2ccc(Cl)cc2c1-c1ccccc1]

As used herein, a compound “inhibits” an activity if the compound reduces the desired activity by at least 10% relative to the activity under the same conditions but lacking only the presence of the compound. The activity may be measured by any reproducible means. The activity may be measured in vitro or in vivo. In some embodiments, compounds used in the methods described herein inhibit a menin activity by at least about 20%, by at least about 25%, by at least about 30%, by at least about 40%, by at least about 50%, by at least about 60%, by at least about 70%, by at least about 75%, by at least about 80%, by at least about 90%, by about 95%, by about 98%, or by about 99% or more. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

The term “IC₅₀” as used herein, is intended to refer to the concentration of a binding molecule capable of interacting with sufficient quantities of menin molecules to produce an effect on approximately 50% of the treated cells.

As used herein, “binding affinity” is represented by the Kᵢ value which is the inhibition constant correlated with the concentration of compound required to occupy the 50% of the total number (Bₘₐₓ) of the molecules of interest, e.g., menin. The lower the Kᵢ value, the higher the binding affinity. The binding affinity may be measured by any reproducible means. The binding affinity may be measured in vitro or in vivo. In some embodiments, compounds used in the methods described herein bind menin with a binding affinity of 10 μM or less, 1 μM or less, 900 nM or less, 800 nM or less, 700 nM or less, 600 nM or less, 500 nM or less, 400 nM or less, 300 nM or less, 200 nM or less, 100 nM or less, or 50 nM or less or any range of the foregoing values.

In some embodiments, the cancer being treated is a cancer in an endocrine organ (e.g., parathyroid, pituitary, and pancreas). For example, the cancer being treated is multiple endocrine neoplasia. In some embodiments, the cancer being treated is a cancer is associated with a chromosomal translocation of the Mixed Lineage Leukemia (Mll) gene. In some embodiments, the cancer being treated a leukemia (e.g., acute myeloid leukemia and acute lymphoblastic leukemia). In some embodiments, skin cancer, skin lesions or malignancies are not being screened or tested. In some embodiments, the diabetes being treated is Type I diabetes. In some embodiments, the diabetes being treated is Type II diabetes.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, when referring to a measurable value such as an amount, a temporal duration, and the like, may encompass variations of ±20% or ±10%, more preferably ±5%, even more preferably ±1%, and still more preferably ±0.1% from the specified value, as such variations are appropriate to perform the disclosed methods.

The term “subject” or “patient” includes mammals, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like). In a preferred embodiment, the subject is human.

“Treating”, includes any effect, e.g., lessening, reducing, modulating, or eliminating, that results in the improvement of the condition, disease, disorder, etc. “Treating” or “treatment” of a disease state includes: (1) inhibiting the disease state, i.e., arresting the development of the disease state or its clinical symptoms or (2) relieving the disease state, i.e., causing temporary or permanent regression of the disease state or its clinical symptoms.

“Preventing” means causing the clinical symptoms of the disease state not to develop, i.e., inhibiting the onset of disease, in a subject that may be exposed to or predisposed to the disease state, but does not yet experience or display symptoms of the disease state.

The methods of “treatment” employ administration to a subject, in need of such treatment, a composition of the present invention, for example, a subject afflicted a disease or disorder, or a subject who ultimately may acquire such a disease or disorder, in order to prevent, cure, delay, reduce the severity of, or ameliorate one or more symptoms of the disorder or recurring disorder, or in order to prolong the survival of a subject beyond that expected in the absence of such treatment.

“Disease state” means any disease, disorder, condition, symptom, or indication.

An “effective amount” of a compound is the quantity which, when administered to a subject having a disease or disorder, results in regression of the disease or disorder in the subject. Thus, for example, for a cell proliferation disorder an effective amount of a compound of the invention is the quantity which, when administered to a subject having a cell proliferation disorder, results in regression of cell growth in the subject. The amount of the compound to be administered to a subject will depend on the particular disorder, the mode of administration, coadministered compounds, if any, and the characteristics of the subject, such as general health, other diseases, age, sex, genotype, body weight and tolerance to drugs. The skilled artisan will be able to determine appropriate dosages depending on these and other factors.

“A therapeutically effective amount” means the amount of a compound that, when administered to a mammal, e.g., a human, for treating a disease, is sufficient to effect such treatment for the disease. The “therapeutically effective amount” will vary depending on the compound, the disease and its severity and the age, weight, etc., of the mammal to be treated.

A therapeutically effective amount of one or more of the compounds of the invention can be formulated with a pharmaceutically acceptable carrier for administration to a human or an animal. Accordingly, the compounds or the formulations can be administered, for example, via oral, parenteral, or topical routes, to provide an effective amount of the compound.

The term “prophylactically effective amount” means an effective amount of a compound or compounds of the invention that is administered to prevent or reduce the risk of a disease state.

“Pharmacological effect” as used herein encompasses effects produced in the subject that achieve the intended purpose of a therapy.

The term “substituted,” as used herein, means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced. Keto substituents are not present on aromatic moieties. Ring double bonds, as used herein, are double bonds that are formed between two adjacent ring atoms (e.g., C═C, C═N, or N═N).

When any variable (e.g., R⁴) occurs more than one time in any constituent or formula for a compound, its definition at each occurrence is independent of its definition at every other occurrence. Thus, for example, if a group is shown to be substituted with 0-3 R⁴ moieties, then the group may optionally be substituted with up to three R⁴ moieties and R⁴ at each occurrence is selected independently from the definition of R⁴. Also, combinations of substituents and/or variables are permissible, but only if such combinations result in stable compounds.

When an atom or chemical moiety is followed by a subscripted numeric range (e.g., C₁₋₆), it will be appreciated that this is meant to encompass each number within the range as well as all intermediate ranges. For example, “C₁₋₆ alkyl” is meant to include alkyl groups with 1, 2, 3, 4, 5, 6, 1-6, 1-5, 1-4, 1-3, 1-2, 2-6, 2-5, 2-4, 2-3, 3-6, 3-5, 3-4, 4-6, 4-5, and 5-6 carbons.

As used herein, “alkyl” is intended to include both branched and straight-chain saturated aliphatic hydrocarbon groups having the specified number of carbon atoms. For example, C₁₋₆ alkyl is intended to include C₁, C₂, C₃, C₄, C₅, and C₆ alkyl groups. Examples of alkyl include, but are not limited to, methyl, ethyl, n-propyl, i-propyl, n-butyl, isobutyl s-butyl, t-butyl, n-pentyl, s-pentyl, neopentyl and n-hexyl. In certain embodiments, a straight chain or branched chain alkyl has six or fewer carbon atoms in its backbone (e.g., C₁-C₆ for straight chain, C₃-C₆ for branched chain), and in another embodiment, a straight chain or branched chain alkyl has four or fewer carbon atoms. Likewise, cycloalkyls have from three to eight carbon atoms in their ring structure, and in other embodiments, cycloalkyls have five or six carbons in the ring structure. Most preferred is (C₁-C₆)alkyl, particularly ethyl, methyl, isopropyl, isobutyl, n-pentyl, n-hexyl and cyclopropylmethyl.

As used herein, the term “substituted alkyl” means alkyl as defined above, substituted by one, two or three substituents selected from the group consisting of halogen, —OH, alkoxy, —NH₂, —N(CH₃)₂, —C(═O)OH, trifluoromethyl, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

Unless the number of carbons is otherwise specified, “lower alkyl” includes an alkyl group, as defined above, but having from one to six carbon atoms in its backbone structure. “Lower alkenyl” and “lower alkynyl” have chain lengths of 2-6 carbon atoms.

“Alkenyl” includes unsaturated aliphatic groups analogous in length and possible substitution to the alkyls described above, but that contain at least one double bond. For example, the term “alkenyl” includes straight-chain alkenyl groups (e.g., ethenyl, propenyl, butenyl, pentenyl, hexenyl, heptenyl, octenyl, nonenyl, decenyl), branched-chain alkenyl groups, cycloalkenyl (e.g., alicyclic) groups (e.g., cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. In certain embodiments, a straight chain or branched chain alkenyl group has six or fewer carbon atoms in its backbone (e.g., C₂-C₆ for straight chain, C₃-C₆ for branched chain). Likewise, cycloalkenyl groups may have from three to eight carbon atoms in their ring structure, and in some embodiments, cycloalkenyl groups have five or six carbons in the ring structure. The term “C₂-C₆” includes alkenyl groups containing two to six carbon atoms. The term “C₃-C₆” includes alkenyl groups containing three to six carbon atoms.

“Alkynyl” includes unsaturated aliphatic groups analogous in length and possible substitution to the alkyls described above, but which contain at least one triple bond. For example, “alkynyl” includes straight-chain alkynyl groups (e.g., ethynyl, propynyl, butynyl, pentynyl, hexynyl, heptynyl, octynyl, nonynyl, decynyl), branched-chain alkynyl groups, and cycloalkyl or cycloalkenyl substituted alkynyl groups. In certain embodiments, a straight chain or branched chain alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

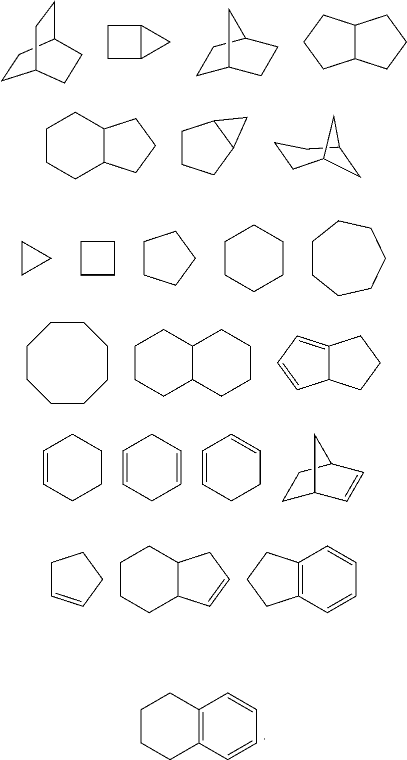

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon double bond or one carbon triple bond.

As used herein, “halo” or “halogen” refers to a fluorine, chlorine, bromine, or iodine atom, preferably, fluorine, chlorine, or bromine, more preferably, fluorine or chlorine. The term “perhalogenated” refers to a moiety wherein all hydrogens are replaced by halogen atoms.

The term “haloalkyl” refers to alkyl moieties having a halogen atom replacing a hydrogen atom on one or more carbons of the hydrocarbon backbone. C₁-C₆ haloalkyl is intended to include a straight chain or branched alkyl having six or fewer carbon atoms in its backbone and a halogen atom replacing a hydrogen atom on one or more carbons of the hydrocarbon backbone.

The term “alkoxy” or “alkoxyl” includes substituted and unsubstituted alkyl, alkenyl, and alkynyl groups covalently linked to an oxygen atom. C₁-C₆ alkoxy refers to moieties having six of few carbon atoms in the hydrocarbon backbone. Examples of alkoxy groups (or alkoxyl radicals) include methoxy, ethoxy, isopropyloxy, propoxy, butoxy, and pentoxy groups. Preferred are (C₁-C₃) alkoxy, particularly ethoxy and methoxy. Examples of substituted alkoxy groups include halogenated alkoxy groups.

The term “hydroxy” or “hydroxyl” includes groups with an —OH or —O⁻.

Also provided herein are methods for treating diabetes in a subject, the method comprising: the step of administering to a subject with diabetes a therapeutically effective amount of a pharmaceutical composition comprising a thienopyrimidine compound (e.g., a compound Formula B) or a pharmaceutically acceptable salt, solvate, hydrate or stereoisomer thereof. In some embodiments, the thienopyrimidine compounds enhance pancreatic β-cell proliferation.

Thienopyrimidine compounds are known in the art and described, for example, in U.S. Patent Application Publication No. 2011/0065690, U.S. Pat. No. 7,300,935 and PCT International Patent Application Publications WO 2012/154009 and WO 2012/030894, which are incorporated by reference herein in their entirety.

In some embodiments, the thienopyrimidine compound is a compound of Formula B:

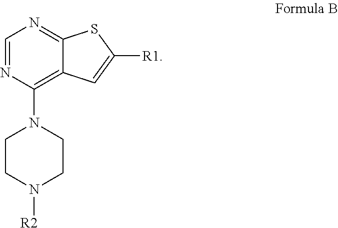

or pharmaceutically acceptable salts or hydrates thereof. In Formula B above, R1 is H, unsubstituted or substituted alkyl, unsubstituted or substituted alkoxy, a halogen (e.g. F, Cl, Br, I, and At), a carbocyclic aromatic ring, a carbocyclic aromatic ring comprising six carbons, a carbocyclic non-aromatic ring, a carbocyclic non-aromatic ring of three to six carbons, a heterocyclic aromatic ring, a five or six member heterocyclic aromatic ring comprising carbon atoms and one or more nitrogen, oxygen and/or sulfur members, an aromatic or non-aromatic ring may be unsubstituted or substituted with alkyl, aryl, halogen, hydrogen bond donor or acceptor, a five or six member heterocyclic non-aromatic ring comprising carbon atoms and one or more nitrogen, oxygen and/or sulfur members, a carbocyclic aromatic or non-aromatic ring fused to the thienopyrimidine ring system, a five or six member carbocyclic aromatic or non-aromatic ring fused to the thienopyrimidine ring system, an aromatic or non-aromatic ring system may be unsubstituted or substituted with alkyl, halogen, hydrogen bond donor or acceptor fused to thienopyrimidine ring system, a five or six member carbocyclic or heterocyclic aromatic ring comprising carbon atoms and one or more nitrogen, oxygen and/or sulfur members fused to another unsubstituted or substituted aromatic ring, or a hydrogen bond donor or a hydrogen bond acceptor. In Formula B above, R2 is H, unsubstituted or substituted alkyl, unsubstituted or substituted alkoxy, a halogen (e.g. F, Cl, Br, I, and At), a carbocyclic aromatic ring, a carbocyclic aromatic ring comprising six carbons, a carbocyclic non-aromatic ring, a carbocyclic non-aromatic ring of three to six carbons, a heterocyclic aromatic ring, a five or six member heterocyclic aromatic ring comprising carbon atoms and one or more nitrogen, oxygen and/or sulfur members, an aromatic or non-aromatic ring may be unsubstituted or substituted with alkyl, aryl, halogen, hydrogen bond donor or acceptor, a five or six member heterocyclic non-aromatic ring comprising carbon atoms and one or more nitrogen, oxygen and/or sulfur members, a carbocyclic aromatic or non-aromatic ring fused to the thienopyrimidine ring system, a five or six member carbocyclic aromatic or non-aromatic ring fused to the thienopyrimidine ring system, an aromatic or non-aromatic ring system may be unsubstituted or substituted with alkyl, halogen, hydrogen bond donor or acceptor fused to thienopyrimidine ring system, a five or six member carbocyclic or heterocyclic aromatic ring comprising carbon atoms and one or more nitrogen, oxygen and/or sulfur members fused to another unsubstituted or substituted aromatic ring, or a hydrogen bond donor or a hydrogen bond acceptor. In some embodiments, R1 and R2 may comprise any substituents which result in compounds which inhibit the interaction of MLL proteins with menin.

In some embodiments, R1 is selected from cyclohexyl, CH₂CH₃, CH₂CH₂CH₃, CH(CH₃)₂, CH₂CH₂CH₂CH₃, CH(CH₃)CH₂CH₃, CF₃, CH₂CF₃, CH₂CH₂CF₃, CH₂CH₂CH₂CF₃, Ph, CH₂Ph, CH₂CH₂Ph, CH₂CH₂CH₂Ph, or CH₂CH₂CH₂CH₂Ph.

In some embodiments, R2 is selected from a functional group set forth in the table below.

R2

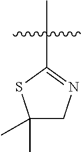 [SMILES: *C1=NCC(C)(C)S1]

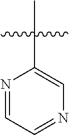 [SMILES: *c1cnccn1]

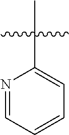 [SMILES: *c1ccccn1]

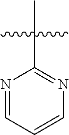 [SMILES: *c1ncccn1]

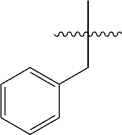 [SMILES: *Cc1ccccc1]

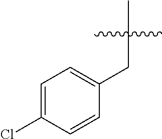 [SMILES: *Cc1ccc(Cl)cc1]

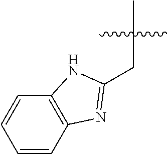 [SMILES: *Cc1nc2ccccc2[nH]1]

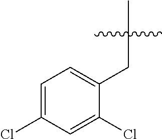 [SMILES: *Cc1ccc(Cl)cc1Cl]

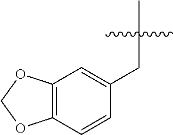 [SMILES: *Cc1ccc2c(c1)OCO2]

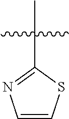 [SMILES: *c1nccs1]

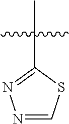 [SMILES: *c1nncs1]

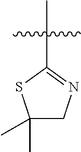 [SMILES: *C1=NCC(C)(C)S1]

In some embodiments, the compound of Formula B is a compound of Formula C below:

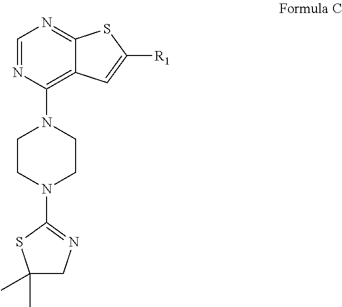

or pharmaceutically acceptable salts or hydrates thereof. In some embodiments, in Formula C above, R1 is cyclohexyl, CH₂CH₃, CH₂CH₂CH₃, CH(CH₃)₂, CH₂CH₂CH₂CH₃, CH(CH₃)CH₂CH₃, CF₃, CH₂CF₃, CH₂CH₂CF₃, CH₂CH₂CH₂CF₃, Ph, CH₂Ph, CH₂CH₂Ph, CH₂CH₂CH₂Ph, or CH₂CH₂CH₂CH₂Ph. In a preferred embodiment, in Formula C above, R1 is CH₂CF₃ (i.e., compound MI-2-2).

In some embodiments, the thienopyrimidine compounds inhibit the interaction of MLL proteins (e.g. MLL fusion proteins) with menin. In a preferred embodiment, the thienopyrimidine compound is MI-2-2 compound which inhibits the bivalent menin-MLL interaction. MI-2-2 compound is known in the art. See e.g., Shi et al., 2012, Blood, vol. 120(23), pages 4461-4469.

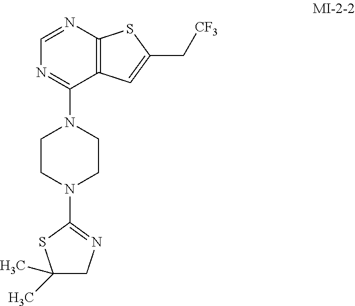 [SMILES: CC1(C)CN=C(N2CCN(c3ncnc4sc(CC(F)(F)F)cc34)CC2)S1]

In some embodiments, at least one of R² and R³ of Formula A is an unsubstituted C₁-C₆ alkyl or a C₁-C₆ alkyl substituted with at least one halogen. In some embodiments, both R² and R³ of Formula A are selected from an unsubstituted C₁-C₆ alkyl or a C₁-C₆ alkyl substituted with at least one halogen. For example, R² and/or R³ is an unsubstituted methyl or ethyl or is a methyl or ethyl substituted with at least one halogen.

In some embodiments, at least one of R¹ and R⁴ of Formula A is NR^{a}R^{b}. In some embodiments, both R¹ and R⁴ of Formula A are each independently NR^{a}R^{b}. In some of the foregoing embodiments, at least one R^{a} and/or R^{b} is a hydrogen. For example, all R^{a} and R^{b} are hydrogen.

In some embodiments, at least one of R¹ and R⁴ of Formula A is a hydroxyl. In some embodiments, both R¹ and R⁴ of Formula A are hydroxyls.

In some embodiments, at least one of R¹ and R⁴ of Formula A is a linear or branched C₁-C₆ alkyl, linear or branched C₂-C₆ alkenyl, linear or branched C₂-C₆ alkynyl, C₃-C₈ cycloalkyl or C₃-C₈ cycloalkenyl. For example, both R¹ and R⁴ are selected from a C₁-C₆ alkyl.

In some embodiments, at least one of R¹ and R⁴ of Formula A is a C₁-C₆ alkoxy. In some embodiments, both R¹ and R⁴ of Formula A is selected from a C₁-C₆ alkoxy.

In some embodiments, each halogen substitution in Formula A is independently selected from fluorine, chlorine, bromine, or iodine. In some embodiment, all halogen substitutions in Formula A are fluorines. In some embodiment, all halogen substitutions in Formula A are chlorines. In some embodiment, all halogen substitutions in Formula A are bromines. In some embodiment, all halogen substitutions in Formula A are iodines.

In some embodiments, the compound of Formula A binds to menin with an affinity of about 10 μM or less. For example, the compound binds to menin with nanomolar affinity.

In some embodiments, the compound of Formula A inhibits the interaction of menin and an MLL protein by at least about 10%, by at least about 20%, by at least about 25%, by at to least about 30%, by at least about 40%, by at least about 50%, by at least about 60%, by at least about 70%, by at least about 75%, by at least about 80%, by at least about 90%, by about 95%, by about 98%, or by about 99% or more. In some of the foregoing embodiments, the MLL protein is a wild-type protein. In some of the foregoing embodiments, the MLL protein is an MLL fusion protein.

In some embodiments, the compound of Formula A is Compound I (N,N′-bis(4-aminophenyl)-N,N′-dimethylethylenediamine).

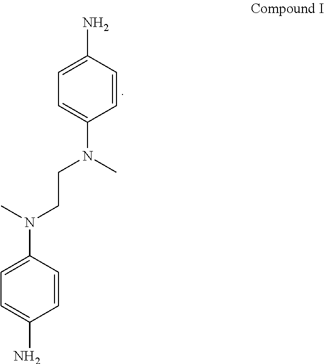 [SMILES: CN(CCN(C)c1ccc(N)cc1)c1ccc(N)cc1]

In some embodiments, the compound of Formula A is Compound II:

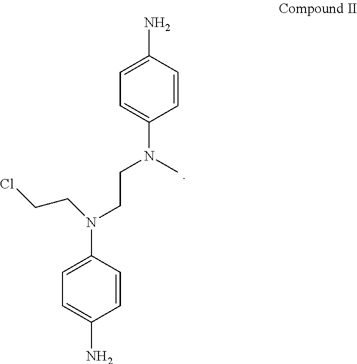 [SMILES: CN(CCN(CCCl)c1ccc(N)cc1)c1ccc(N)cc1]

In some embodiments, the thienopyrimidine compounds reverse and/or inhibit the oncogenic (e.g. leukemogenic) effects of MLL-fusion proteins, and/or MLL/menin and MLL fusion proteins/menin interactions. In some embodiments, the thienopyrimidine compounds prevent or treat leukemia.

In another aspect, methods for screening a potential diabetes therapeutic are provided, the methods include the steps of: growing pancreatic β-cells in the presence of a compound suspected of being a diabetes therapeutic, growing pancreatic β-cells in the presence of a reference compound, determining the rate of growth of the pancreatic β-cells in the presence of said compound and the rate of growth of the pancreatic β-cells in the presence of the reference compound and comparing the growth rate of the pancreatic β-cells, wherein a higher rate of growth of pancreatic β-cells in the presence of said compound than in the presence of said reference compound is indicative of a diabetes therapeutic, wherein said reference compound is a compound of Formula B or a pharmaceutically acceptable salt, solvate, hydrate or stereoisomer thereof.

In another aspect, methods for identifying a compound that enhances pancreatic β-cell proliferation are provided, the methods include the steps of: growing β-cells in the presence of a compound suspected of enhancing pancreatic β-cell proliferation, growing pancreatic β-cells in the presence of a reference compound, determining the rate of growth of the pancreatic β-cells in the presence of said compound and the rate of growth of the pancreatic β-cells in the presence of the reference compound and comparing the growth rate of the pancreatic β-cells, wherein a higher rate of growth of pancreatic β-cells in the presence of said compound than in the presence of said reference compound is indicative of a compound that enhances pancreatic β-cell proliferation, wherein said reference compound is a compound of Formula B or a to pharmaceutically acceptable salt, solvate, hydrate or stereoisomer thereof.

The compounds described herein may exist in their isomeric forms, for example, but are not limited to, stereoisomers, chiral isomers, and geometric isomers.

“Isomerism” means compounds that have identical molecular formulae but that differ in the nature or the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images are termed “enantiomers”, or sometimes optical isomers. A carbon atom bonded to four nonidentical substituents is termed a “chiral center”.

“Chiral isomer” means a compound with at least one chiral center. It has two enantiomeric forms of opposite chirality and may exist either as an individual enantiomer or as a mixture of enantiomers. A mixture containing equal amounts of individual enantiomeric forms of opposite chirality is termed a “racemic mixture”. A compound that has more than one chiral center has 2ⁿ⁻¹ enantiomeric pairs, where n is the number of chiral centers. Compounds with more than one chiral center may exist as either an individual diastereomer or as a mixture of diastereomers, termed a “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al, Angew. Chem. Inter. Edit. 1966, 5, 385; errata 511; Cahn et al, Angew. Chem. 1966, 78, 413; Cahn and Ingold, J. Chem. Soc. 1951 (London), 612; Cahn et al, Experientia 1956, 12, 81; Cahn, J., Chem. Educ. 1964, 41, 1 16).

“Geometric Isomer” means the diastereomers that owe their existence to hindered rotation about double bonds. These configurations are differentiated in their names by the to prefixes cis and trans, or Z and E, which indicate that the groups are on the same or opposite side of the double bond in the molecule according to the Cahn-Ingold-Prelog rules.

Further, the structures and other compounds discussed in this application include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

Additionally, the compounds described herein, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutical composition” is a formulation containing the inhibitors in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient (e.g., a formulation of the disclosed inhibitor) in a unit dose of composition is an effective amount and is varied according to the particular test or screen involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. In some embodiments, the route of administration is oral. Dosage forms for the topical or transdermal administration of a compound of this invention include powders, sprays, ointments, pastes, creams, lotions, gels, solutions, patches and inhalants. In some embodiments, the active compound is mixed under sterile conditions with a pharmaceutically acceptable carrier, and with any preservatives, buffers, or propellants that are required.

“Pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used herein includes both one and more than one such excipient.

As used herein, “pharmaceutically acceptable salts” refer to derivatives of the compounds wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The invention also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs), of the same salt.

The pharmaceutically acceptable salts of the invention can be synthesized from a parent compound that contains a basic or acidic moiety by conventional chemical methods. Generally, such salts can be prepared by the reaction of the free acid or base forms of the parent compounds with a stoichiometric amount of the appropriate base or acid in water or in an organic solvent, or in a mixture of the two; non-aqueous media like ether, ethyl acetate, ethanol, isopropanol, or acetonitrile can be used. Lists of suitable salts are found in Remington's Pharmaceutical Sciences, 18th ed. (Mack Publishing Company, 1990). For example, salts can include, but are not limited to, the hydrochloride and acetate salts of the aliphatic amine-containing, hydroxyl amine-containing, and imine-containing compounds of the present invention.

Compounds of the invention can also be prepared as esters, for example pharmaceutically acceptable esters. For example a carboxylic acid function group in a compound can be converted to its corresponding ester, e.g., a. methyl, ethyl, or other ester. Also, an alcohol group in a compound can be converted to its corresponding ester, e.g., an acetate, propionate, or other ester.

Compounds of the invention can also be prepared as prodrugs, for example pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.) the compounds of the present invention can be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a subject. Prodrugs may be prepared by modifying functional groups present in the compound in such a way that the modifications are cleaved, either in routine manipulation or in vivo, to the parent compound. Prodrugs include compounds of the invention wherein a hydroxy, amino, sulfhydryl, carboxy, or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates, and benzoate derivatives) and carbamates (e.g., N,N-dimethylaminocarbonyl) of hydroxy functional groups, esters groups (e.g. ethyl esters, morpholinoethanol esters) of carboxyl functional groups, N-acyl derivatives (e.g. N-acetyl)N-Mannich bases, Schiff bases and enaminones of amino functional groups, oximes, acetals, ketals and enol esters of ketone and aldehyde functional groups in compounds of Formula I, and the like, See Bundegaard, H. “Design of Prodrugs” p1-92, Elesevier, New York-Oxford (1985).

“Stable compound” and “stable structure” are meant to indicate a compound that is sufficiently robust to survive isolation to a useful degree of purity from a reaction mixture, and formulation into an efficacious therapeutic agent.

Type I diabetes begins, generally, before the clinical manifestations of the disease. It starts with the progressive destruction of β-cells in the pancreas. These cells normally produce insulin. The reduction of insulin response to glucose can be measured during this period, however. Ultimately, there is massive (>90%) destruction of β-cells in the islets of Langerhans. During the early stages of the disease and beyond, type I diabetes is characterized by the infiltration of pancreatic islets by macrophages and lymphocytes (helper and killer). The macrophage infiltration prompts the infiltration of small lymphocytes.

Type II diabetes is characterized, inter alia, by insulin resistance, i.e., a failure of the normal metabolic response of peripheral tissues to the action of insulin. In other words, insulin resistance refers to a condition where the circulating insulin produces a subnormal biological response. In clinical terms, insulin resistance is present when normal or elevated blood glucose levels persist in the face of normal or elevated levels of insulin. The hyperglycemia associated with Type II diabetes is often reversed or ameliorated by diet or weight loss sufficient to restore the sensitivity of the peripheral tissues to insulin. Type II diabetes mellitus may also encompass hyperglycemia in the presence of higher than normal levels of plasma insulin. Progression of Type II diabetes mellitus is associated with increasing concentrations of blood glucose and coupled with a relative decrease in the rate of glucose-induced insulin secretion. Thus, in late-stage Type II diabetes mellitus, an insulin deficiency persists.

In certain embodiments, the compositions of the invention are administered (either concurrently or separately) for the treatment of diabetes in conjunction with other therapeutic agents for diabetes. Representative agents that can be used in combination with the compositions of the invention are agents used to treat diabetes such as insulin and insulin analogs (e.g., LysPro insulin); GLP-1 (7-37) (insulinotropin) and GLP-1 (7-36)-NH₂; biguanides: metformin, phenformin, buformin; α2-antagonists and imidazolines: midaglizole, isaglidole, deriglidole, idazoxan, efaroxan, fluparoxan; sulfonylureas and analogs: chlorpropamide, glibenclamide, tolbutamide, tolazamide, acetohexamide, glypizide, glimepiride, repaglinide, meglitinide; other insulin secretagogues: linogliride, A-4166; glitazones: ciglitazone, pioglitazone, englitazone, troglitazone, darglitazone, rosiglitazone; PPAR-gamma agonists; fatty acid oxidation inhibitors: clomoxir, etomoxir; α-glucosidase inhibitors: acarbose, miglitol, emiglitate, voglibose, MDL-25,637, camiglibose, MDL-73,945; β-agonists: BRL 35135, BRL 37344, Ro 16-8714, ICI D7114, CL 316,243; phosphodiesterase inhibitors: L-386,398; lipid-lowering agents: benfluorex; antiobesity agents: fenfluramine; vanadate and vanadium complexes (e.g., NAGLIVAN®)) and peroxovanadium complexes; amylin antagonists; glucagon antagonists; gluconeogenesis inhibitors; somatostatin analogs and antagonists; antilipolytic agents: nicotinic acid, acipimox, WAG 994. Also contemplated for use in combination with the compositions of the invention are pramlintide acetate (SYMLIN™), AC2993, glycogen phosphorylase inhibitor and nateglinide. Any combination of agents can be administered as described hereinabove. In one embodiment, dipeptidypeptidase-4 (DPP-4) inhibitors; vildagliptin (LAF 237), sitagliptin (MK 0431), ZP10, or combination thereof are given with the compositions and methods of the invention as oral anti-diabetic medication as well.

The methods for the treatment of cancers according to embodiments of the invention also may be used in combination with the treatment of a cancer or cell proliferation disorder with one or more of anti-cancer treatments such as surgery, radiation therapy, immunotherapy and/or one or more anti-cancer agents selected from the group consisting of anti-proliferative agents, agents that modulate the metabolism of cancer cells, cytotoxic agents, cytostatic agents, and chemotherapeutic agents and salts and derivatives thereof. According to certain embodiments, the treatment of a cancer or cell proliferation disorder in a therapy with any one of the drugs selected from a group consisting of an alkaloid, an alkylating agent, an antitumor antibiotic, an antimetabolite, a Bcr-Abl tyrosine kinase inhibitor, a nucleoside analogue, a multidrug resistance reversing agent, a DNA binding agent, microtubule binding drug, a toxin and a DNA antagonist. Those of skill in the art will recognize the chemotherapeutic agents classified into one or more particular classes of chemotherapeutic agents described above.

As used herein, the term “cell proliferative disorder” refers to conditions in which unregulated or abnormal growth, or both, of cells can lead to the development of an unwanted condition or disease, which may or may not be cancerous. Exemplary cell proliferative disorders of the invention encompass a variety of conditions wherein cell division is deregulated.

As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms.

Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue. A cell proliferative disorder includes a precancer or a precancerous condition. A cell proliferative disorder includes cancer. Preferably, the methods provided herein are used to treat or alleviate a symptom of cancer. The term “cancer” includes solid tumors, as well as, hematologic tumors and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer.

Exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, uringary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Sézary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute to myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplasia/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

As used herein, the term “severity” is meant to describe the potential of cancer to transform from a precancerous, or benign, state into a malignant state. Alternatively, or in addition, severity is meant to describe a cancer stage, for example as described below, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

For example, A cancer or pre-malignant legion can be staged according to the American Joint Committee on Cancer (AJCC) TNM classification system, where the tumor (T) has been assigned a stage of TX, T1, Tlmic, Tla, Tib, Tic, T2, T3, T4, T4a, T4b, T4c, or T4d; and where the regional lymph nodes (N) have been assigned a stage of NX, NO, N1, N2, N2a, N2b, N3, N3a, N3b, or N3c; and where distant metastasis (M) can be assigned a stage of MX, M0, or M1. Alternatively, A cancer or pre-malignant legion can be staged according to an American Joint Committee on Cancer (AJCC) classification as Stage I, Stage IIA, Stage IIB, Stage IIIA, Stage IIIB, Stage IIIC, or Stage IV. A cancer or pre-malignant lesion can also be assigned a grade according to an AJCC classification as Grade GX (e.g., grade cannot be assessed), Grade 1, Grade 2, Grade 3 or Grade 4. A cancer or pre-malignant lesion can in another example be staged according to an AJCC pathologic classification (pN) of pNX, pNO, PNO (I−), PNO (I+), PNO (mol−), PNO (mol+), PN1, PNl(mi), PNl a, PNlb, PNlc, pN2, pN2a, pN2b, pN3, pN3a, pN3b, or pN3c.

The compositions are administered orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the inhibitor is administered orally. One skilled in the art will recognize the advantages of certain routes of administration.

The dosage regimen utilizing the inhibitors is selected in accordance with a variety of factors including type, species, age, weight, sex and medical condition of the patient; the severity of the condition to be treated; the route of administration; the renal and hepatic function of the patient; and the particular inhibitor employed. An ordinarily skilled physician or veterinarian can readily determine and prescribe the effective amount of the drug required.

Techniques for formulation and administration of compositions of the invention can be found in Remington: the Science and Practice of Pharmacy, 19^{th} edition, Mack Publishing Co., Easton, Pa. (1995). The inhibitors described herein are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

In some embodiments, the compound is prepared for oral administration, wherein the inhibitors is combined with a suitable solid or liquid carrier or diluent to form capsules, tablets, pills, powders, syrups, solutions, suspensions and the like. The tablets, pills, capsules, and the like contain from about 1 to about 99 weight percent of the active ingredient and a binder such as gum tragacanth, acacias, corn starch or gelatin; excipients such as dicalcium phosphate; a disintegrating agent such as corn starch, potato starch or alginic acid; a lubricant such as magnesium stearate; and/or a sweetening agent such as sucrose, lactose, saccharin, xylitol, and to the like. When a dosage unit form is a capsule, it often contains, in addition to materials of the above type, a liquid carrier such as a fatty oil.

In some embodiments, various other materials are present as coatings or to modify the physical form of the dosage unit. For instance, in some embodiments, tablets are coated with shellac, sugar or both. In some embodiments, a syrup or elixir contains, in addition to the inhibitor, sucrose as a sweetening agent, methyl and propylparabens as preservatives, a dye and a flavoring such as cherry or orange flavor, and the like.

For some embodiments relating to parental administration, the inhibitors, can be combined with sterile aqueous or organic media to form injectable solutions or suspensions. In some embodiments, injectable compositions are aqueous isotonic solutions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.1 to 75%, in another embodiment, the compositions contain about 1 to 50%, of the active ingredient.

For example, injectable solutions are produced using solvents such as an oil or aqueous propylene glycol, as well as aqueous solutions of water-soluble pharmaceutically-acceptable salts of the inhibitor. In some embodiments, dispersions are prepared in glycerol, liquid polyethylene glycols and mixtures thereof in oils. Under ordinary conditions of storage and use, these preparations contain a preservative to prevent the growth of microorganisms. The terms “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

For rectal administration, suitable pharmaceutical compositions are, for example, topical preparations, suppositories or enemas. Suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.1 to 75%, in another embodiment, compositions contain about 1 to 50%, of the inhibitor.

In some embodiments, the inhibitors are formulated for delivery by pulmonary administration, e.g., administration of an aerosol formulation containing the inhibitor from, for example, a manual pump spray, nebulizer or pressurized metered-dose inhaler. In some embodiments, suitable formulations of this type also include other agents, such as antistatic agents, to maintain the disclosed compounds as effective aerosols.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a headspace representing greater than about 15%) of the total volume of the canister. Often, the polymer intended for pulmonary administration is dissolved, suspended or emulsified in a mixture of a solvent, surfactant and propellant. The mixture is maintained under pressure in a canister that has been sealed with a metering valve.

For nasal administration, either a solid or a liquid carrier can be used. The solid carrier includes a coarse powder having particle size in the range of, for example, from about 20 to about 500 microns and such formulation is administered by rapid inhalation through the nasal passages. In some embodiments where the liquid carrier is used, the formulation is administered as a nasal spray or drops and includes oil or aqueous solutions of the active ingredients.

All sequence citations, accession numbers, references, patents, patent applications, scientific publications or other documents cited are hereby incorporated by reference.

In the specification, the singular forms also include the plural, unless the context clearly dictates otherwise. Unless defined otherwise, all technical and scientific terms used herein have the same meaning as commonly understood by one of ordinary skill in the art to which this invention belongs. In the case of conflict, the present specification will control.

All percentages and ratios used herein, unless otherwise indicated, are by weight.

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### EXAMPLES

### Example 1

### Identification of Compounds that Inhibit Menin MLL Interaction

### In Silico Screening

In silico screening of 140,000+ small-molecule compounds was performed to identify those compounds that would inhibit the interaction of the menin protein and the MLL peptide using the solved co-crystal structure (Huang et al. (2012) Nature 482:542). The top 80 ranked compounds based on energy released upon binding were obtained from the National Cancer Institute's Developmental Therapeutics Program for further in vitro testing. Each of the approved in silico compounds (ISCs) was dissolved in DMSO and serially diluted on 384-well plates.

### Menin Protein Purification

His-Sumo-Menin-Δ460-519 plasmid was obtained from Dr. Ming Lei at the University of Michigan. Protein was transformed into Rosetta DE3-pLysS E. coli (Novagen) and expressed with 0.1 mM IPTG overnight (16 hours) at 20° C. Bacterial lysates were incubated with Ni-NTA beads (Qiagen) to extract His-Sumo-Menin-Δ460-519, and protein was eluted with 200 mM imidazole (Sigma). Protein fractions were pooled and concentrated using an Ultra YM-10 CENTRIPREP® spin filter column (Millipore), and subjected to FPLC through a Hi-Load 16/60 SUPERDEX® 200 column.

### MLL Peptide Synthesis

MLL peptides were synthesized by the Yale University Keck Facility in New Haven, Conn., and dissolved in DMSO. Sequences for each peptide are as follows:

MLL-FITC:

(SEQ ID NO. 1)

fluorescein-RWRFPARPGTGRRG-amide
MLL-WT:

(SEQ ID NO. 2)

biotin-RWRFPARPGTGRRG-amide
MLL-Mut:

(SEQ ID NO. 3)

biotin-MAHSCAWAFPGSGSCAWAFP-amide

### In Vitro Fluorescence Polarization

In 384-well plates, fluorescence polarization buffer (40 mM HEPES pH 7.9, 150 mM NaCl, 0.01% Triton X-100, 10 mM 2-mercaptoethanol) is added to each well containing MLL-FITC peptide (1.5 nM), His-Sumo-Menin-A460-519 (5 nM), and the dilutions of each ISC (concentrations ranging from 35 nM to 100 μM). For control wells, the ISC was replaced with either 1 μM MLL-WT or 1 μM MLL-Mut to serve as positive and negative controls, respectively. Plates were read immediately for millipolarization (mP) values against a blank (wells containing only buffer) on an ENVISION® Multilabel Reader (Perkin Elmer) after the components were loaded.

### Calculation of Normalized IC₅₀ Values

Blanked mP values were normalized on a percent scale where 0% represents no inhibition (based on the average of the 1 μM MLL-Mut control well mP values) and 100% represents high inhibition (based on the average of the 1 μM MLL-WT control well mP values). ISCs which were calculated to fit a log-IC₅₀ non-linear regression on this scale were examined and selected for further cell-based testing.

### Cell Dosing, Growth Inhibition, and Gene Expression

ISCs were tested in leukemic cell lines (AT-1, a mouse MLL-AF9 transformed cell line; THP-1, a human MLL-AF9 transformed cell line) at 10 μM and 50 μM concentrations, with DMSO vehicle as a control. Cells were counted two and four days post-treatment for signs of growth inhibition or toxicity. Four days after treatment, cells were harvested and RNA was isolated for cDNA production for quantitative real-time PCR to examine selected gene expression.

### Results

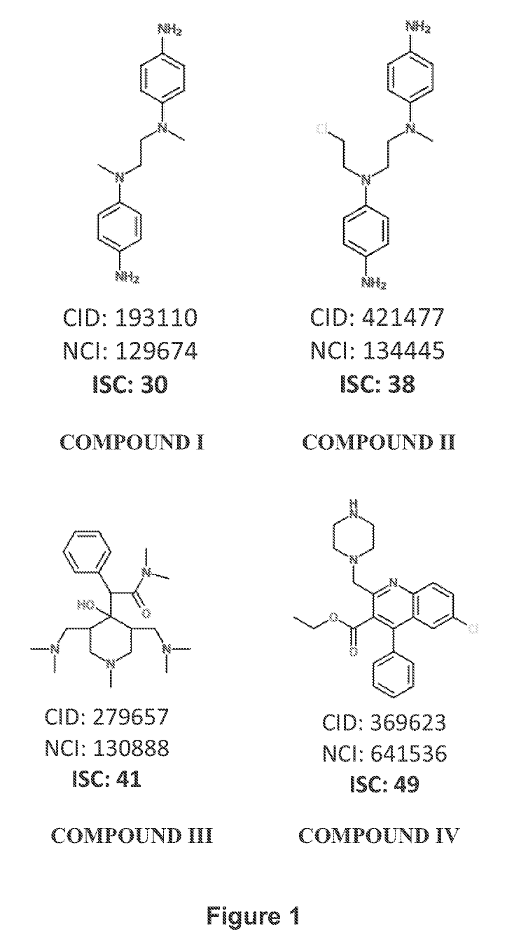

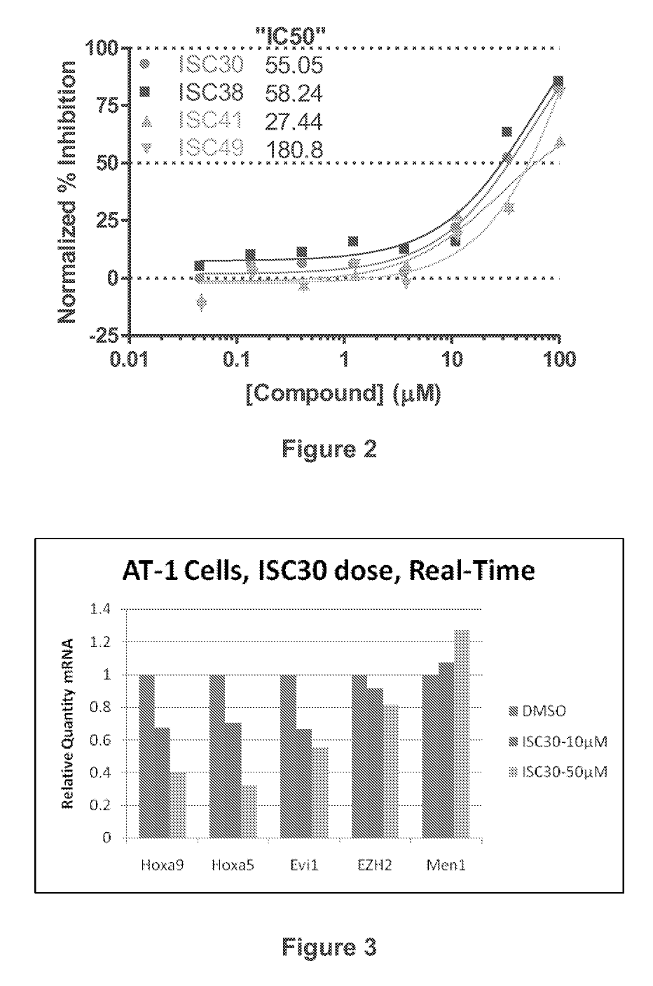

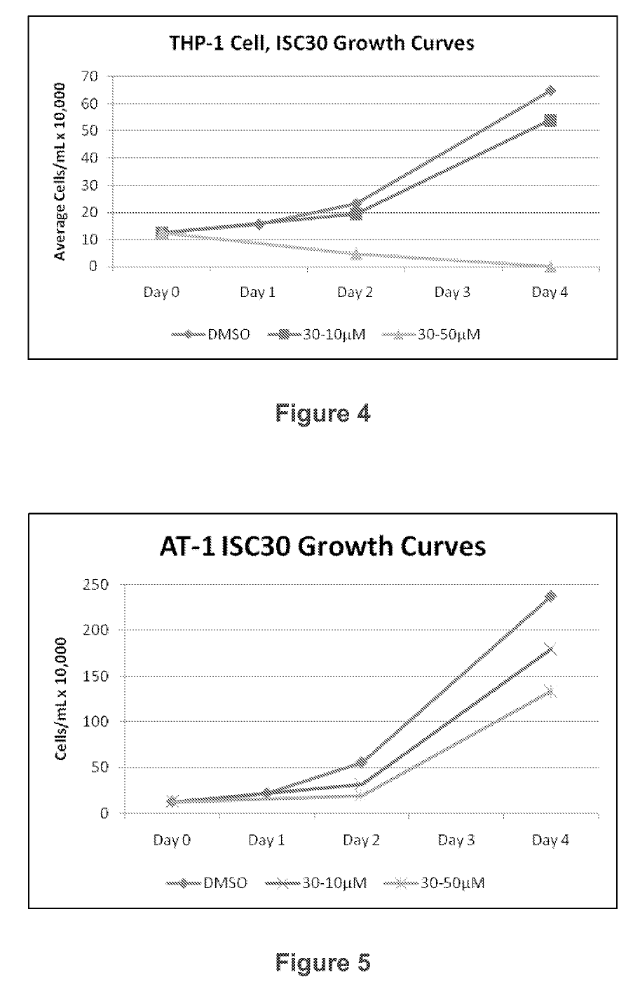

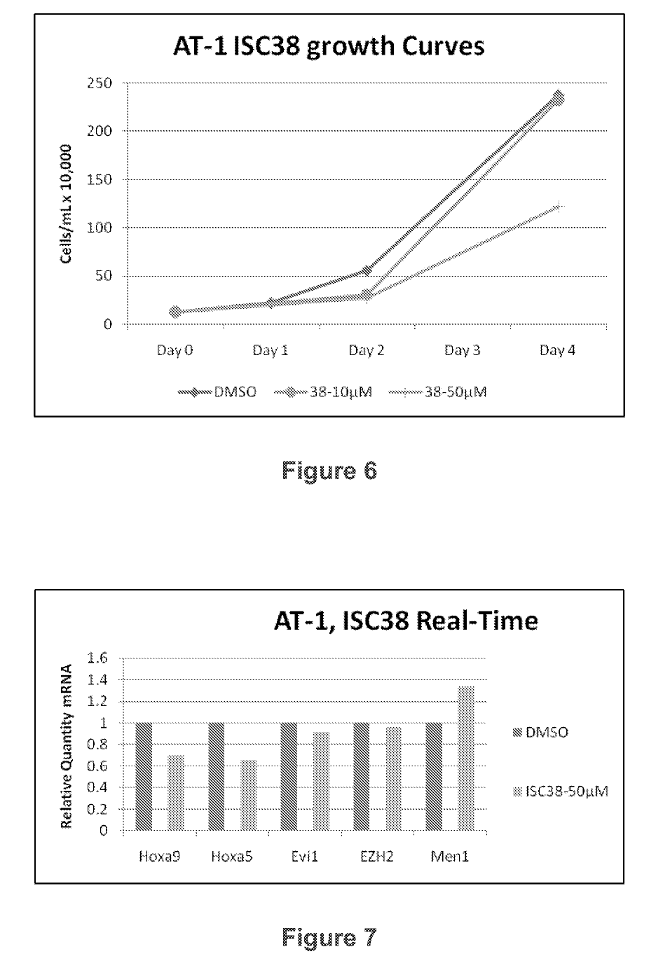

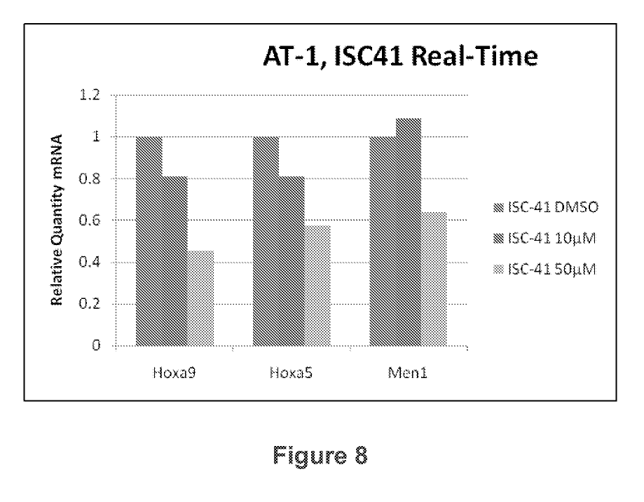

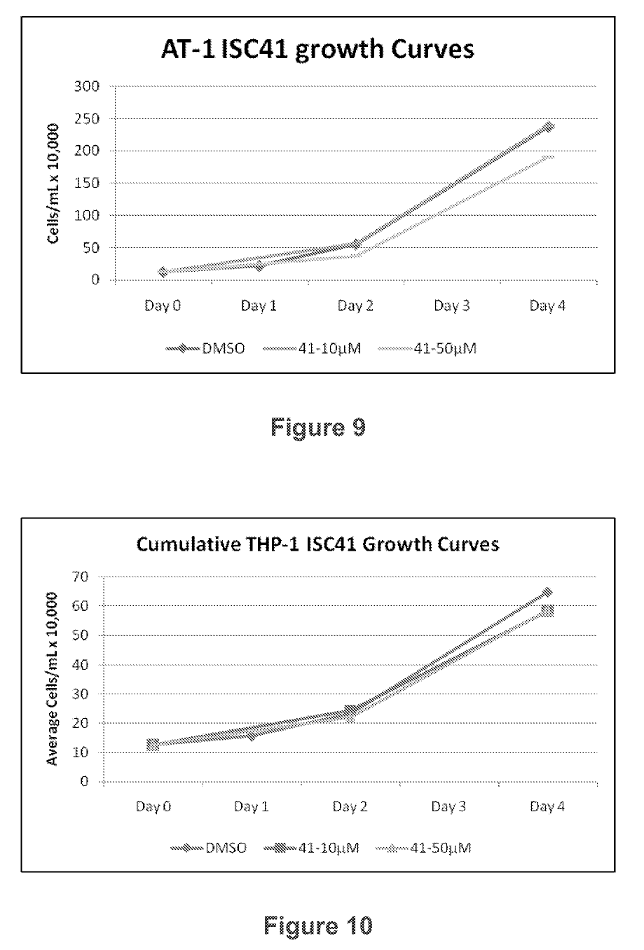

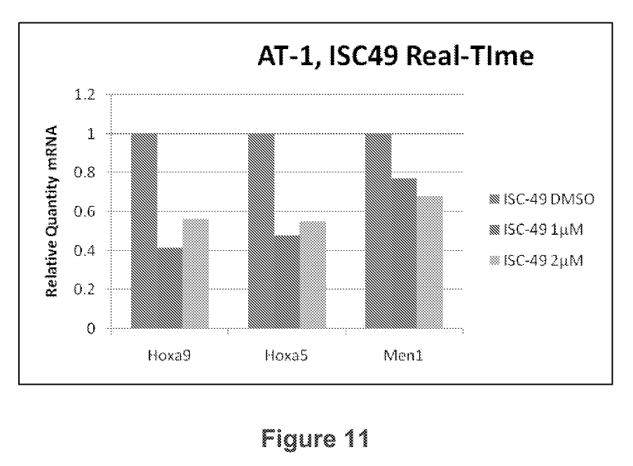

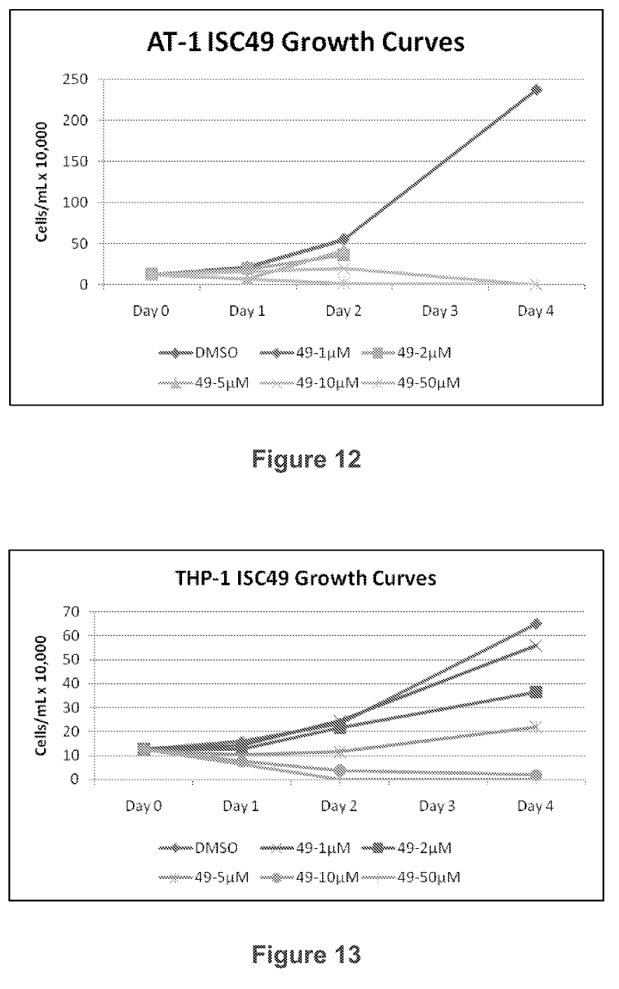

The top 80 ranked compounds based on energy released upon binding were obtained from the National Cancer Institute's Developmental Therapeutics Program for further in vitro testing. Of these compounds, six compounds were found to inhibit the interaction between a His-Sumo-MeninΔ460-519 protein construct and a fluorescein-labeled MLL peptide in a fluorescence polarization assay (see FIG. 2). Of these six compounds, four, whose structures are shown in FIG. 1, have been tested further. These compounds have been tested in both human and mouse MLL leukemia cell line models. All four compounds were tested in the AT-1 cell line, a mouse MLL-AF9 transformed cell line (see FIGS. 5, 6, 9, and 12). Three of the four compounds (all but Compound II) were tested in the human THP-1 cell line, a human MLL-AF9 transformed cell line (see FIGS. 4, 10, and 13). Based on these tests, these compounds were found to inhibit cell proliferation to varying degrees. All four compounds have also been shown (see FIGS. 3, 7, 8, and 11) to down-regulate gene targets, such as Hoxa9 and Hoxa5, which depend on the menin-MLL interaction.

Thus, these compounds have been demonstrated to inhibit the interaction between menin and its MLL substrate, as well as to reduce menin-MLL-dependent downsteam gene targets.

### Example 2

### Menin Inhibition Stimulates Cell Proliferation and GLP-1 Receptor Expression

Diabetes involves an insufficiency of pancreatic beta cell mass. Type 1 diabetes (T1D) results from an absolute insufficiency of beta cell mass, while type 2 diabetes (T2D) involves a relative insufficiency. Pancreatic islet transplantation is the only current means to restore beta cell mass. However, it has several limitations including graft rejection and limited supply of donor islets. Pharmacological approaches to increase beta cell mass in-situ or in in-vitro cultures of islets offer a means of overcoming these limitations. In this regard, we have reported that excision of multiple endocrine neoplasia type 1 (MEN1) leads to reversal of glucose intolerance in several murine diabetes models including streptozotozcin induced diabetes, high-fat diet mice, and obese (db/db) mice. This improvement of glucose tolerance is explained in part by the enhancement of pancreatic β-cell proliferation and increased plasma insulin levels.

We have resolved the co-crystal structure of menin along with mixed lineage leukemia (MLL). We identified a hydrophobic pocket in menin that interacts with MLL and has potential to bind several other proteins. Surprisingly and unexpectedly, we have found that pharmacological inhibition of this binding pocket in menin increases beta cell proliferation and mass, as well as increases the expression of GLP1 receptor.

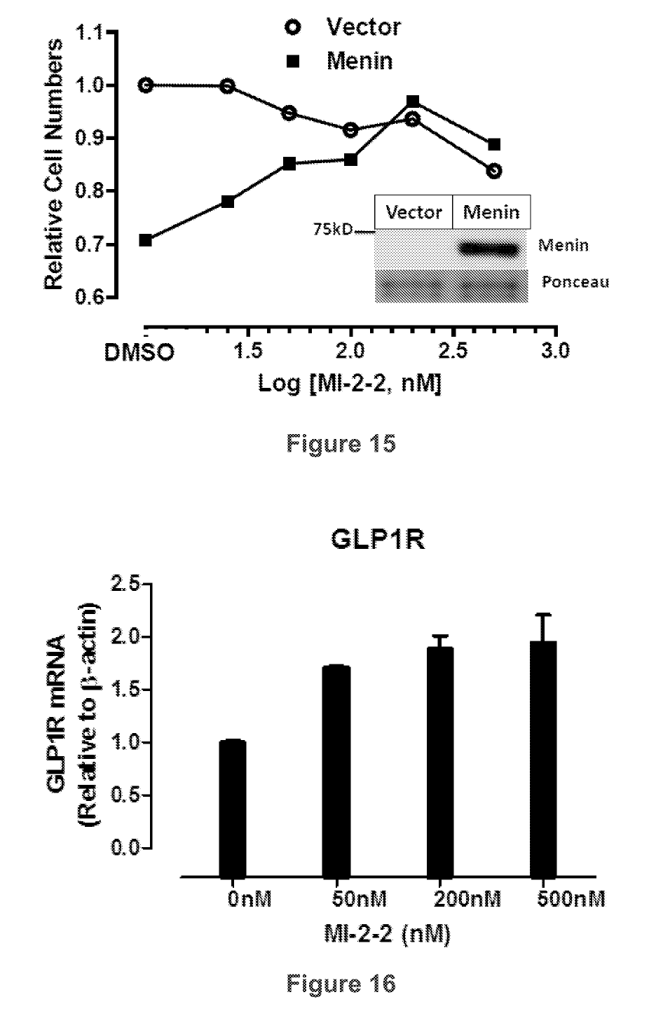

We have found that the menin inhibitor MI-2-2, described by Shi et al (Blood 2012), is able to stimulate cell growth in mouse embryonic fibroblasts (MEFs) that were either menin null (vector) or menin null cells complemented with exogenous menin (menin). Furthermore, MI-2-2 increased cell growth in menin MEFs in a concentration dependent manner; but it did not do so in vector MEFs (FIG. 15). Vector and menin-expressing MEFs (FIG. 15, inset) were seeded at 0.25×10⁶ cells/10 cm dish on day 0, treated with either DMSO or increasing concentrations of MI-2-2 from day 1 as indicated, and counted after six days of treatment. Thus, inhibition of menin antagonizes menin mediated suppression of cell proliferation.

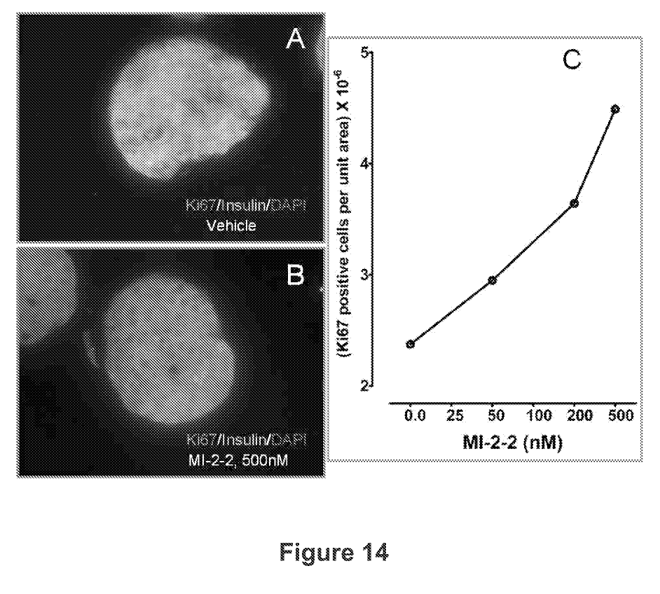

The ability of MI-2-2 to stimulate cell growth was further tested in murine islets isolated from C57BL/6J mice (Jackson Laboratories, Bar Harbor, Me.). Treatment of murine islets with MI-2-2 for 2 days increased the number of proliferating cells in a concentration dependent manner, as measured by increased Ki67 positive cells per unit area upon treatment with MI-2-2 (FIG. 14).

Menin inhibition also increases expression of GLP1 receptor, which is the receptor mediating the pro-islet incretin's ability to reduce blood glucose (FIG. 16). Human islets were procured from the Islet Transplantation Center, University of Pennsylvania and cultured in a humidified incubator with 5% CO₂ at 37° C. After 24 hours, the islets were treated with vehicle control (DMSO) or varying concentrations of MI-2-2 (50 nM, 200 nM or 500 nM) for 2 days. The islets were harvested in TRIzol® (Life Technologies, Grand Island, N.Y.) and stored at −80° C. RNA was isolated using the RNeasy Mini Kit (Qiagen, Germantown, Md.) and was reverse transcribed to cDNA using the SuperScript® III Reverse Transcriptase (Life Technologies, Grand Island, N.Y.). cDNA was quantitated by real time quantitative PCR using QuantiTect SYBR® Green (Qiagen, Germantown, Md.) on a 7500 Fast Real time PCR system (AppliedBiosystems, Life Technologies, Grand Island, N.Y.) and normalized using β-actin as endogenous control. As shown in FIG. 16, inhibition of menin by MI-2-2 increases expression of GLP1 receptor (relative to β-actin expression) by about 2-fold.

Currently the availability of human pancreatic islets is a limiting factor to restoring beta cell mass. MI-2-2 can be used to stimulate pancreatic beta cell proliferation in-vivo, ex-vivo or in-vitro. Therefore, MI-2-2 can be used to overcome the need for islet transplantation or at least to maximize currently available islet sources by increasing their mass in-vitro prior to transplantation or in vivo post transplantation. Also, MI-2-2 can be used as a pharmacological tool or drug to induce pancreatic beta cell regeneration to treat type 2 diabetes. In summary, these findings show that pharmacological inhibition of menin can stimulate cell proliferation, which can be employed to induce pancreatic beta cell proliferation to treat diabetes.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of treating diabetes in a subject, the method comprising: the step of administering to a subject with diabetes a therapeutically effective amount of a pharmaceutical composition of a compound of Formula B or a pharmaceutically acceptable salt, solvate, hydrate or stereoisomer thereof: 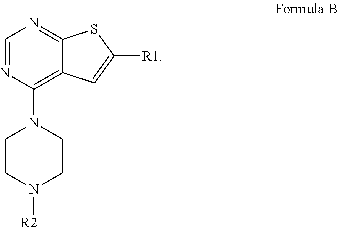
wherein each of R1 and R2 comprises a substituent which results in a compound that inhibits the interaction of MLL proteins with menin.

2. The method of claim 1, wherein R1 is H, alkyl, alkoxy, a halogen, a carbocyclic aromatic ring, a carbocyclic aromatic ring comprising six carbons, a carbocyclic non-aromatic ring, a carbocyclic non-aromatic ring of three to six carbons, a heterocyclic aromatic ring, a five or six member heterocyclic aromatic ring comprising carbon atoms and one or more nitrogen, oxygen and/or sulfur members, any aromatic or non-aromatic ring non-substituted or substituted with alkyl, aryl, halogen, hydrogen bond donor or acceptor, a five or six member heterocyclic non-aromatic ring comprising carbon atoms and one or more nitrogen, oxygen and/or sulfur members, a carbocyclic aromatic or non-aromatic ring fused to the thienopyrimidine ring system, a five or six member carbocyclic aromatic or non-aromatic ring fused to the thienopyrimidine ring system, any aromatic or non-aromatic ring system non-substituted or substituted with alkyl, halogen, hydrogen bond donor or acceptor fused to thienopyrimidine ring system, a five or six member carbocyclic or heterocyclic aromatic ring comprising carbon atoms and one or more nitrogen, oxygen and/or sulfur members fused to another aromatic ring substituted or non-substituted, or a hydrogen bond donor or a hydrogen bond acceptor; and R2 is H, alkyl, alkoxy, a halogen, a carbocyclic aromatic ring comprising six carbons, a carbocyclic non-aromatic ring of three to six carbons, a five or six member heterocyclic aromatic ring comprising carbon atoms and one or more nitrogen, oxygen and/or sulfur members, a five or six member heterocyclic non-aromatic ring comprising carbon atoms and one or more nitrogen, oxygen and/or sulfur members, any aromatic or non-aromatic ring non-substituted or substituted with alkyl, aryl, halogen, hydrogen bond donor or acceptor, a five or six member carbocyclic or heterocyclic aromatic ring comprising carbon atoms and one or more nitrogen, oxygen and/or sulfur members fused to another aromatic ring, a hydrogen bond donor or a hydrogen bond acceptor.

3. The method of claim 1, wherein R1 is cyclohexyl, CH₂CH₃, CH₂CH₂CH₃, CH(CH₃)₂, CH₂CH₂CH₂CH₃, CH(CH₃)CH₂CH₃, CF₃, CH₂CF₃, CH₂CH₂CF₃, CH₂CH₂CH₂CF₃, Ph, CH₂Ph, CH₂CH₂Ph, CH₂CH₂CH₂Ph, or CH₂CH₂CH₂CH₂Ph and R2 comprises a functional group selected from the table below.
R2

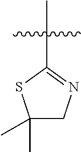 [SMILES: *C1=NCC(C)(C)S1]

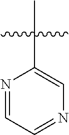 [SMILES: *c1cnccn1]

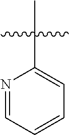 [SMILES: *c1ccccn1]

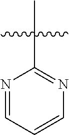 [SMILES: *c1ncccn1]

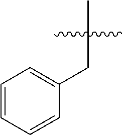 [SMILES: *Cc1ccccc1]

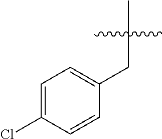 [SMILES: *Cc1ccc(Cl)cc1]

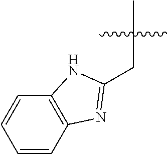 [SMILES: *Cc1nc2ccccc2[nH]1]

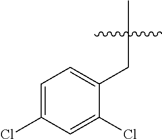 [SMILES: *Cc1ccc(Cl)cc1Cl]

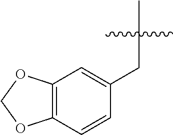 [SMILES: *Cc1ccc2c(c1)OCO2]

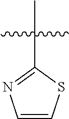 [SMILES: *c1nccs1]

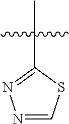 [SMILES: *c1nncs1]

4. The method claim 1, wherein said compound is a compound of Formula C or a pharmaceutically acceptable salt, solvate, hydrate or stereoisomer thereof: 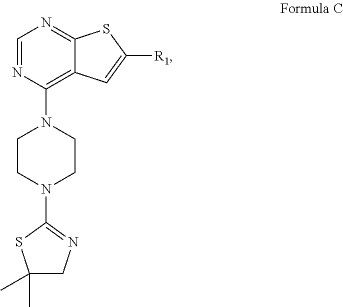
wherein R1 is cyclohexyl, CH₂CH₃, CH₂CH₂CH₃, CH(CH₃)₂, CH₂CH₂CH₂CH₃, CH(CH₃)CH₂CH₃, CF₃, CH₂CF₃, CH₂CH₂CF₃, CH₂CH₂CH₂CF₃, Ph, CH₂Ph, CH₂CH₂Ph, CH₂CH₂CH₂Ph, or CH₂CH₂CH₂CH₂Ph.

5. The method of claim 4, wherein R1 is CH₂CF₃.

6. The method according to claim 1, wherein said compound binds to menin with an affinity of about 1 μM or less.

7. The method according to claim 6, wherein said compound binds to menin with an affinity of about 100 nM or less.

8. The method according to any one of claims 1-7, wherein the subject has Type I diabetes.

9. The method according to any one of claims 1-7, wherein the subject has Type II diabetes.

10. A method for enhancing pancreatic β-cell proliferation in a subject, the method comprising: the step of administering to a subject with diabetes a therapeutically effective amount of a pharmaceutical composition of a compound of Formula B or a pharmaceutically acceptable salt, solvate, hydrate or stereoisomer thereof: 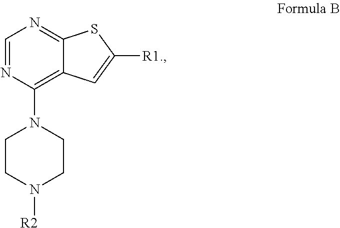
wherein each of R1 and R2 comprises a substituent which results in a compound that inhibits the interaction of MLL proteins with menin.

11. The method of claim 10, wherein R1 is H, alkyl, alkoxy, a halogen, a carbocyclic aromatic ring, a carbocyclic aromatic ring comprising six carbons, a carbocyclic non-aromatic ring, a carbocyclic non-aromatic ring of three to six carbons, a heterocyclic aromatic ring, a five or six member heterocyclic aromatic ring comprising carbon atoms and one or more nitrogen, oxygen and/or sulfur members, any aromatic or non-aromatic ring non-substituted or substituted with alkyl, aryl, halogen, hydrogen bond donor or acceptor, a five or six member heterocyclic non-aromatic ring comprising carbon atoms and one or more nitrogen, oxygen and/or sulfur members, a carbocyclic aromatic or non-aromatic ring fused to the thienopyrimidine ring system, a five or six member carbocyclic aromatic or non-aromatic ring fused to the thienopyrimidine ring system, any aromatic or non-aromatic ring system non-substituted or substituted with alkyl, halogen, hydrogen bond donor or acceptor fused to thienopyrimidine ring system, a five or six member carbocyclic or heterocyclic aromatic ring comprising carbon atoms and one or more nitrogen, oxygen and/or sulfur members fused to another aromatic ring substituted or non-substituted, or a hydrogen bond donor or a hydrogen bond acceptor; and R2 is H, alkyl, alkoxy, a halogen, a carbocyclic aromatic ring comprising six carbons, a carbocyclic non-aromatic ring of three to six carbons, a five or six member heterocyclic aromatic ring comprising carbon atoms and one or more nitrogen, oxygen and/or sulfur members, a five or six member heterocyclic non-aromatic ring comprising carbon atoms and one or more nitrogen, oxygen and/or sulfur members, any aromatic or non-aromatic ring non-substituted or substituted with alkyl, aryl, halogen, hydrogen bond donor or acceptor, a five or six member carbocyclic or heterocyclic aromatic ring comprising carbon atoms and one or more nitrogen, oxygen and/or sulfur members fused to another aromatic ring, a hydrogen bond donor or a hydrogen bond acceptor.

12. The method of claim 10, wherein R1 is cyclohexyl, CH₂CH₃, CH₂CH₂CH₃, CH(CH₃)₂, CH₂CH₂CH₂CH₃, CH(CH₃)CH₂CH₃, CF₃, CH₂CF₃, CH₂CH₂CF₃, CH₂CH₂CH₂CF₃, Ph, CH₂Ph, CH₂CH₂Ph, CH₂CH₂CH₂Ph, or CH₂CH₂CH₂CH₂Ph and R2 comprises a functional group selected from the table below.
R2

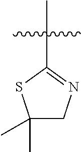 [SMILES: *C1=NCC(C)(C)S1]

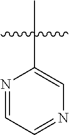 [SMILES: *c1cnccn1]

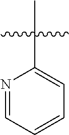 [SMILES: *c1ccccn1]

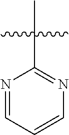 [SMILES: *c1ncccn1]

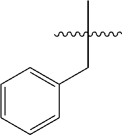 [SMILES: *Cc1ccccc1]

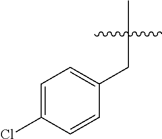 [SMILES: *Cc1ccc(Cl)cc1]

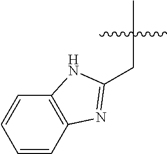 [SMILES: *Cc1nc2ccccc2[nH]1]

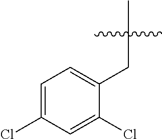 [SMILES: *Cc1ccc(Cl)cc1Cl]

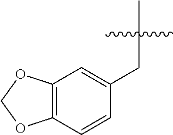 [SMILES: *Cc1ccc2c(c1)OCO2]

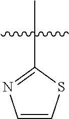 [SMILES: *c1nccs1]

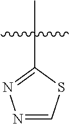 [SMILES: *c1nncs1]

13. The method claim 10, wherein said compound is a compound of Formula C or a pharmaceutically acceptable salt, solvate, hydrate or stereoisomer thereof: 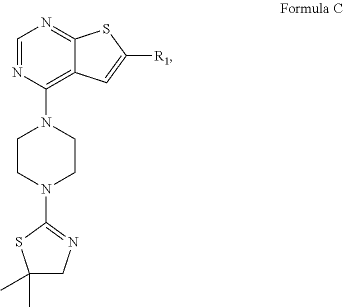
wherein R1 is cyclohexyl, CH₂CH₃, CH₂CH₂CH₃, CH(CH₃)₂, CH₂CH₂CH₂CH₃, CH(CH₃)CH₂CH₃, CF₃, CH₂CF₃, CH₂CH₂CF₃, CH₂CH₂CH₂CF₃, Ph, CH₂Ph, CH₂CH₂Ph, CH₂CH₂CH₂Ph, or CH₂CH₂CH₂CH₂Ph.

14. The method of claim 13, wherein R1 is CH₂CF₃.

15. The method according to claim 10, wherein said compound binds to menin with an affinity of about 1 μM or less.

16. The method according to claim 15, wherein said compound binds to menin with an affinity of about 100 nM or less.

17. The method according to any one of claims 10-16, wherein the subject has Type I diabetes.

18. The method according to any one of claims 10-16, wherein the subject has Type II diabetes.

19. A method for treating cancer in a subject comprising the step of administering to a subject with cancer a therapeutically effective amount of a pharmaceutical composition of a compound of Formula A or a pharmaceutically acceptable salt, solvate, hydrate or stereoisomer thereof: 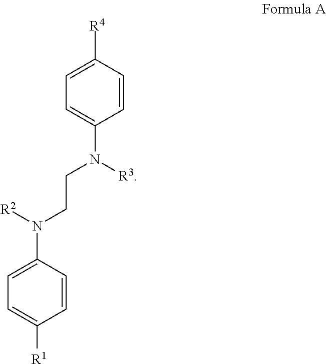
wherein R¹ and R⁴ are each independently selected from the group consisting of hydrogen, halogen, NR^{a}R^{b}, hydroxyl, linear or branched C₁-C₆ alkyl, linear or branched C₂-C₆ alkenyl, linear or branched C₂-C₆ alkynyl, C₃-C₈ cycloalkyl, C₃-C₈ cycloalkenyl, and C₁-C₆alkoxy;
wherein said alkyls, alkenyls, alkynyls, cycloalkyls, cycloalkenyls and alkoxys of R¹ and R⁴ are unsubstituted or substituted with at least one halogen;
wherein R^{a} and R^{b} are each independently selected from group consisting of hydrogen, linear or branched C₁-C₄ alkyl, linear or branched C₂-C₄ alkenyl, C₂-C₄ alkynyl, C₃-C₆ cycloalkyl, and C₃-C₆ cycloalkenyl;
wherein said alkyls, alkenyls, alkynyls, cycloalkyls, cycloalkenyls of R^{a} and R^{b} are unsubstituted or substituted with at least one halogen;
wherein R² and R³ are each independently selected from group consisting of hydrogen, linear or branched C₁-C₆ alkyl, linear or branched C₂-C₆ alkenyl, linear or branched C₂-C₆ alkynyl, C₃-C₈ cycloalkyl, and C₃-C₈ cycloalkenyl;
wherein said alkyls, alkenyls, alkynyls, cycloalkyls, cycloalkenyls of R² and R³ are unsubstituted or substituted with at least one halogen.

20. The method of claim 19, wherein at least one of R² and R³ is an unsubstituted C₁-C₆ alkyl or a C₁-C₆ alkyl substituted with at least one halogen.

21. The method of claim 20, wherein both R² and R³ are each independently selected from an unsubstituted C₁-C₆ alkyl or a C₁-C₆ alkyl substituted with at least one halogen.

22. The method according to claim 21, wherein at least one of R² and R³ is an unsubstituted methyl or ethyl or is a methyl or ethyl substituted with at least one halogen.

23. The method of claim 22, wherein both R² and R³ are each independently selected from an unsubstituted methyl or ethyl or is a methyl or ethyl substituted with at least one halogen.

24. The method according to claim 19, wherein at least one of R¹ and R⁴ is NR^{a}R^{b}.

25. The method according to claim 24, wherein both R¹ and R⁴ are each independently NR^{a}R^{b}.

26. The method according to claim 25, wherein at least one of R^{a} and R^{b} is a hydrogen.

27. The method of claim 26, wherein all R^{a} and R^{b} are hydrogen.

28. The method according to any one of claims 19-27, wherein at least one of R¹ and R⁴ is a hydroxyl.

29. The method according to any one of claims 19-27, wherein at least one of R¹ and R⁴ is a linear or branched C₁-C₆ alkyl, linear or branched C₂-C₆ alkenyl, linear or branched C₂-C₆ alkynyl, C₃-C₈ cycloalkyl or C₃-C₈ cycloalkenyl.

30. The method of claim 29, wherein at least one of R¹ and R⁴ is a C₁-C₆ alkyl.

31. The method according to any one of claims 19-27, wherein at least one of R¹ and R⁴ is a C₁-C₆ alkoxy.

32. The method according to claim 19, wherein all halogen substitutions are chlorines.

33. The method according to claim 19, wherein said compound binds to menin with an affinity of about 10 μM or less.

34. The method according to claim 33, wherein said compound binds to menin with nanomolar affinity.

35. The method according to claim 19, wherein said compound inhibits the interaction of menin and an MLL protein by at least 50%.

36. The method according to claim 35, wherein said MLL protein is a wild-type protein.

37. The method according to claim 35, wherein said MLL protein is an MLL fusion protein.

38. The method according to claim 19, wherein said compound is Compound I:

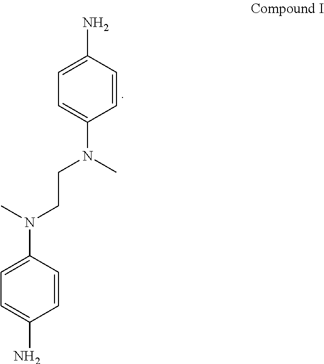 [SMILES: CN(CCN(C)c1ccc(N)cc1)c1ccc(N)cc1]

39. The method according to claim 19, wherein said compound is Compound II:

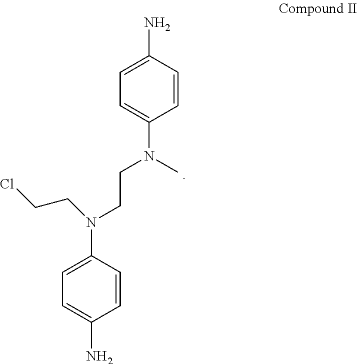 [SMILES: CN(CCN(CCCl)c1ccc(N)cc1)c1ccc(N)cc1]

40. A method for treating cancer in a subject comprising the step of administering to a subject with cancer a therapeutically effective amount of a pharmaceutical composition of Compound III or a pharmaceutically acceptable salt, solvate, hydrate or stereoisomer thereof:

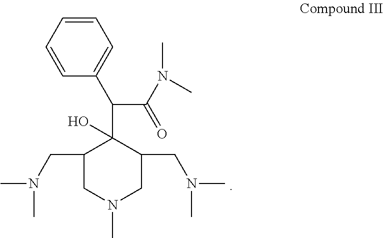

41. A method for treating cancer in a subject comprising the step of administering to a subject with cancer a therapeutically effective amount of a pharmaceutical composition of Compound III or a pharmaceutically acceptable salt, solvate, hydrate or stereoisomer thereof:

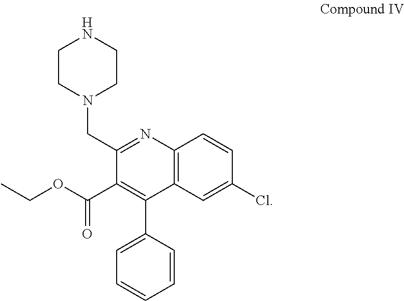 [SMILES: CCOC(=O)c1c(CN2CCNCC2)nc2ccc(Cl)cc2c1-c1ccccc1]

42. The method according to any one of claim 1, 10, 19, 40, or 41, wherein said subject is a human.

43. The method according to any one of claim 19, 40, or 41, wherein said cancer is a cancer in an endocrine organ.

44. The method according to claim 43, wherein said cancer is multiple endocrine neoplasia.

45. The method according to claim 43, wherein said cancer is associated with a chromosomal translocation of the Mixed Lineage Leukemia (Mll) gene.

46. The method according to claim 43, wherein said cancer is a leukemia.

47. The method according to claim 46, wherein said leukemia is acute myeloid leukemia and acute lymphoblastic leukemia.

48. A method of treating diabetes in a subject comprising the step of administering to a subject with a diabetes a therapeutically effective amount of a pharmaceutical composition of a compound of Formula A or a pharmaceutically acceptable salt, solvate, hydrate or stereoisomer thereof: 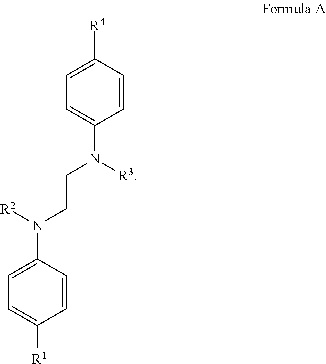
wherein R¹ and R⁴ are each independently selected from the group consisting of hydrogen, halogen, NR^{a}R^{b}, hydroxyl, linear or branched C₁-C₆ alkyl, linear or branched C₂-C₆ alkenyl, linear or branched C₂-C₆ alkynyl, C₃-C₈ cycloalkyl, C₃-C₈ cycloalkenyl, and C₁-C₆alkoxy;
wherein said alkyls, alkenyls, alkynyls, cycloalkyls, cycloalkenyls and alkoxys of R¹ and R⁴ are unsubstituted or substituted with at least one halogen;
wherein R^{a} and R^{b} are each independently selected from group consisting of hydrogen, linear or branched C₁-C₄ alkyl, linear or branched C₂-C₄ alkenyl, C₂-C₄ alkynyl, C₃-C₆ cycloalkyl, and C₃-C₆ cycloalkenyl;
wherein said alkyls, alkenyls, alkynyls, cycloalkyls, cycloalkenyls of R^{a} and R^{b} are unsubstituted or substituted with at least one halogen;
wherein R² and R³ are each independently selected from group consisting of hydrogen, linear or branched C₁-C₆ alkyl, linear or branched C₂-C₆ alkenyl, linear or branched C₂-C₆ alkynyl, C₃-C₈ cycloalkyl, and C₃-C₈ cycloalkenyl;
wherein said alkyls, alkenyls, alkynyls, cycloalkyls, cycloalkenyls of R² and R³ are unsubstituted or substituted with at least one halogen.

49. The method according to claim 48, wherein at least one of R² and R³ is an unsubstituted C₁-C₆ alkyl or a C₁-C₆ alkyl substituted with at least one halogen.

50. The method according to claim 49, wherein both R² and R³ are each independently selected from an unsubstituted C₁-C₆ alkyl or a C₁-C₆ alkyl substituted with at least one halogen.

51. The method according to claim 49, wherein at least one of R² and R³ is an unsubstituted methyl or ethyl or is a methyl or ethyl substituted with at least one halogen.

52. The method according to claim 51, wherein both of R² and R³ are each independently selected from an unsubstituted methyl or ethyl or is a methyl or ethyl substituted with at least one halogen.

53. The method according to claim 48, wherein at least one of R¹ and R⁴ is NR^{a}R^{b}.

54. The method according to claim 49, wherein both R¹ and R⁴ are each independently NR^{a}R^{b}.

55. The method according to claim 53, wherein at least one of R^{a} and R^{b} is a hydrogen.

56. The method according to claim 55, wherein all R^{a} and R^{b} are hydrogen.

57. The method according to any one of claims 48-56, wherein at least one of R¹ and R⁴ is a hydroxyl.

58. The method according to any one of claims 48-56, wherein at least one of R¹ and R⁴ is a linear or branched C₁-C₆ alkyl, linear or branched C₂-C₆ alkenyl, linear or branched C₂-C₆ alkynyl, C₃-C₈ cycloalkyl or C₃-C₈ cycloalkenyl.

59. The method according to claim 58, wherein at least one of R¹ and R⁴ is a C₁-C₆ alkyl.

60. The method according to any one of claims 48-56, wherein at least one of R¹ and R⁴ is a C₁-C₆ alkoxy.

61. The method according to claim 48, wherein all halogen substitutions are chlorines.

62. The method according to claim 48, wherein said compound binds to menin with an affinity of about 10 μM or less.

63. The method according to claim 62, wherein said compound binds to menin with nanomolar affinity.

64. The method according to claim 48, wherein said compound is Compound I:

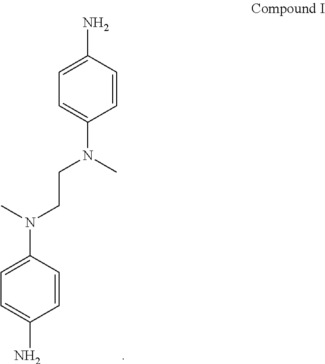 [SMILES: CN(CCN(C)c1ccc(N)cc1)c1ccc(N)cc1]

65. The method according to claim 48, wherein said compound is Compound II:

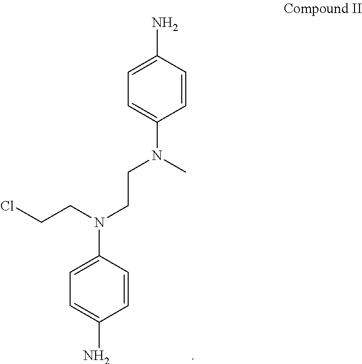 [SMILES: CN(CCN(CCCl)c1ccc(N)cc1)c1ccc(N)cc1]

66. A method for treating diabetes in a subject comprising the step of administering to a subject with a diabetes a therapeutically effective amount of a pharmaceutical composition of Compound III or a pharmaceutically acceptable salt, solvate, hydrate or stereoisomer thereof:

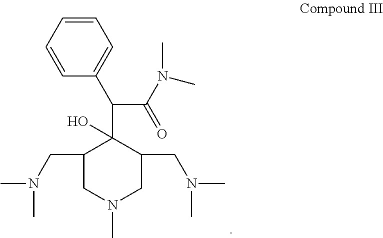

67. A method for treating diabetes in a subject comprising the step of administering to a subject with a diabetes a therapeutically effective amount of a pharmaceutical composition of Compound III or a pharmaceutically acceptable salt, solvate, hydrate or stereoisomer thereof:

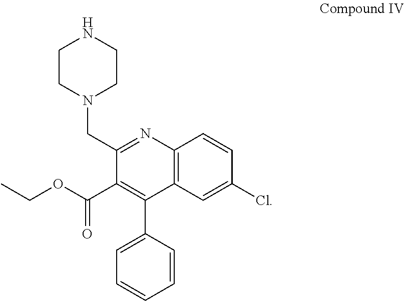 [SMILES: CCOC(=O)c1c(CN2CCNCC2)nc2ccc(Cl)cc2c1-c1ccccc1]

68. The method according to any one of claim 48, 66, or 67, wherein said subject is a human.

69. The method according to any one of claim 48, 66, or 67, wherein said diabetes is a Type-I diabetes.

70. The method according to any one of claim 48, 66, or 67, wherein said diabetes is a Type-I diabetes.

71. A method for inhibiting the growth or proliferation of a cancerous or precancerous cell are provided, the methods include: the step contacting a cancer or precancerous cell with an effective amount of a pharmaceutical composition of a compound of Formula A or a pharmaceutically acceptable salt, solvate, hydrate or stereoisomer thereof: 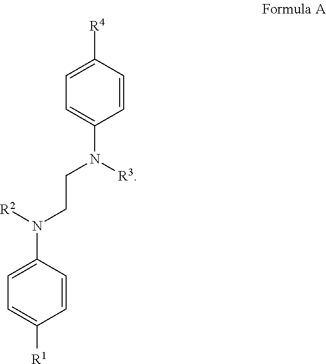
wherein R¹ and R⁴ are each independently selected from the group consisting of hydrogen, halogen, NR^{a}R^{b}, hydroxyl, linear or branched C₁-C₆ alkyl, linear or branched C₂-C₆ alkenyl, linear or branched C₂-C₆ alkynyl, C₃-C₈ cycloalkyl, C₃-C₈ cycloalkenyl, and C₁-C₆alkoxy;
wherein said alkyls, alkenyls, alkynyls, cycloalkyls, cycloalkenyls and alkoxys of R¹ and R⁴ are unsubstituted or substituted with at least one halogen;
wherein R^{a} and R^{b} are each independently selected from group consisting of hydrogen, linear or branched C₁-C₄ alkyl, linear or branched C₂-C₄ alkenyl, C₂-C₄ alkynyl, C₃-C₆ cycloalkyl, and C₃-C₆ cycloalkenyl;
wherein said alkyls, alkenyls, alkynyls, cycloalkyls, cycloalkenyls of R^{a} and R^{b} are unsubstituted or substituted with at least one halogen;
wherein R² and R³ are each independently selected from group consisting of hydrogen, linear or branched C₁-C₆ alkyl, linear or branched C₂-C₆ alkenyl, linear or branched C₂-C₆ alkynyl, C₃-C₈ cycloalkyl, and C₃-C₈ cycloalkenyl;
wherein said alkyls, alkenyls, alkynyls, cycloalkyls, cycloalkenyls of R² and R³ are unsubstituted or substituted with at least one halogen.

72. The method according to claim 71, wherein at least one of R² and R³ is an unsubstituted C₁-C₆ alkyl or a C₁-C₆ alkyl substituted with at least one halogen.

73. The method according to claim 72, wherein both R² and R³ are each independently selected from an unsubstituted C₁-C₆ alkyl or a C₁-C₆ alkyl substituted with at least one halogen.

74. The method according to claim 71, wherein at least one of R² and R³ is an unsubstituted methyl or ethyl or is a methyl or ethyl substituted with at least one halogen.

75. The method according to claim 74, wherein both R² and R³ are each independently selected from an unsubstituted methyl or ethyl or is a methyl or ethyl substituted with at least one halogen.

76. The method according to claim 71, wherein at least one of R¹ and R⁴ is NR^{a}R^{b}.

77. The method according to claim 76, wherein both R¹ and R⁴ are each independently NR^{a}R^{b}.

78. The method according to claim 76, wherein at least one of R^{a} R^{b} is a hydrogen.

79. The method according to claim 77, wherein all R^{a} and R^{b} are hydrogen.

80. The method according to any one of claims 71-79, wherein at least one of R¹ and R⁴ is a hydroxyl.

81. The method according to any one of claims 71-79, wherein at least one of R¹ and R⁴ is a linear or branched C₁-C₆ alkyl, linear or branched C₂-C₆ alkenyl, linear or branched C₂-C₆ alkynyl, C₃-C₈ cycloalkyl or C₃-C₈ cycloalkenyl.

82. The method according to claim 81, wherein at least one of R¹ and R⁴ is a C₁-C₆ alkyl.

83. The method according to any one of claims 71-79, wherein at least one of R¹ and R⁴ is a C₁-C₆ alkoxy.

84. The method according to claim 71, wherein all halogen substitutions are chlorines.

85. The method according to claim 71, wherein said compound binds to menin with an affinity of about 10 μM or less.

86. The method according to claim 75, wherein said compound binds to menin with nanomolar affinity.

87. The method according to claim 71, wherein said compound inhibits the interaction of menin and an MLL protein by at least 50%.

88. The method according to claim 87, wherein said MLL protein is a wild-type protein.

89. The method according to claim 87, wherein said MLL protein is an MLL fusion protein.

90. The method according to claim 71, wherein said compound is Compound I:

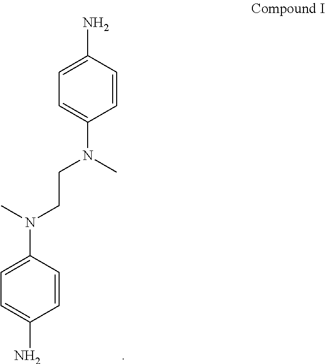 [SMILES: CN(CCN(C)c1ccc(N)cc1)c1ccc(N)cc1]

91. The method according to claim 71, wherein said compound is Compound II:

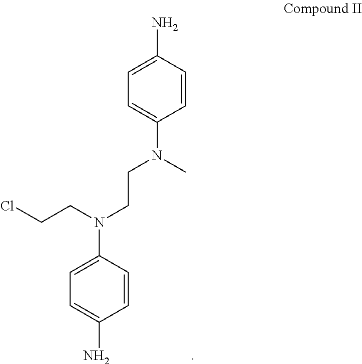 [SMILES: CN(CCN(CCCl)c1ccc(N)cc1)c1ccc(N)cc1]

92. A method for inhibiting the growth or proliferation of a cancerous or precancerous cell are provided, the methods include: the step contacting a cancer or precancerous cell with an effective amount of a pharmaceutical composition of Compound III or a pharmaceutically acceptable salt, solvate, hydrate or stereoisomer thereof:

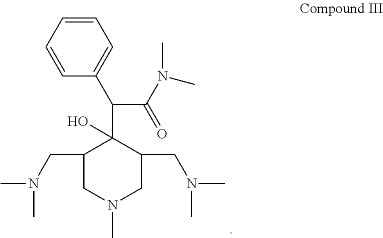

93. A method for inhibiting the growth or proliferation of a cancerous or precancerous cell are provided, the methods include: the step contacting a cancer or precancerous cell with an effective amount of a pharmaceutical composition of Compound III or a pharmaceutically acceptable salt, solvate, hydrate or stereoisomer thereof:

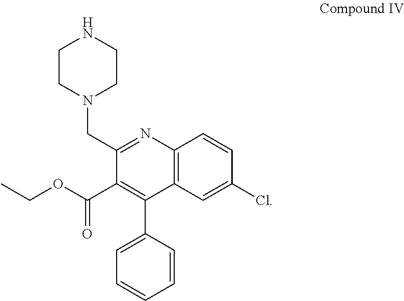 [SMILES: CCOC(=O)c1c(CN2CCNCC2)nc2ccc(Cl)cc2c1-c1ccccc1]

94. The method according to any one of claim 71, 93, or 94, wherein said cancerous or precancerous cell is in vitro cell line.

95. A method for screening a potential cancer therapeutic, comprising the steps of: which comprises: growing cells containing a chromosomal translocation of the Mixed Lineage Leukemia (Mll) gene in the presence of a compound suspected of being a cancer therapeutic, growing said cells in the presence of a reference compound, determining the rate of growth of said cells in the presence of said compound and the rate of growth of said cells in the presence of a reference compound and comparing the growth rate of said cells, wherein a slower rate of growth of said cells in the presence of said compound than in the presence of said reference compound is indicative of a cancer therapeutic, wherein said reference compound is a compound of Formula A or a pharmaceutically acceptable salt, solvate, hydrate or stereoisomer thereof: 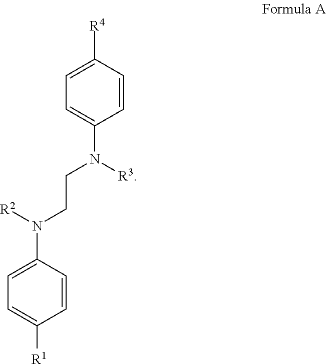
wherein R¹ and R⁴ are each independently selected from the group consisting of hydrogen, halogen, NR^{a}R^{b}, hydroxyl, linear or branched C₁-C₆ alkyl, linear or branched C₂-C₆ alkenyl, linear or branched C₂-C₆ alkynyl, C₃-C₈ cycloalkyl, C₃-C₈ cycloalkenyl, and C₁-C₆alkoxy;
wherein said alkyls, alkenyls, alkynyls, cycloalkyls, cycloalkenyls and alkoxys of R¹ and R⁴ are unsubstituted or substituted with at least one halogen;
wherein R^{a} and R^{b} are each independently selected from group consisting of hydrogen, linear or branched C₁-C₄ alkyl, linear or branched C₂-C₄ alkenyl, C₂-C₄ alkynyl, C₃-C₆ cycloalkyl, and C₃-C₆ cycloalkenyl;
wherein said alkyls, alkenyls, alkynyls, cycloalkyls, cycloalkenyls of R^{a} and R^{b} are unsubstituted or substituted with at least one halogen;
wherein R² and R³ are each independently selected from group consisting of hydrogen, linear or branched C₁-C₆ alkyl, linear or branched C₂-C₆ alkenyl, linear or branched C₂-C₆ alkynyl, C₃-C₈ cycloalkyl, and C₃-C₈ cycloalkenyl;
wherein said alkyls, alkenyls, alkynyls, cycloalkyls, cycloalkenyls of R² and R³ are unsubstituted or substituted with at least one halogen.

96. The method according to claim 95, wherein said compound is Compound I:

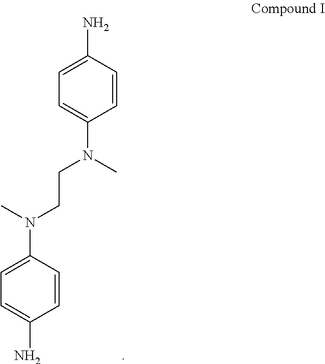 [SMILES: CN(CCN(C)c1ccc(N)cc1)c1ccc(N)cc1]

97. The method according to claim 95, wherein said compound is Compound II:

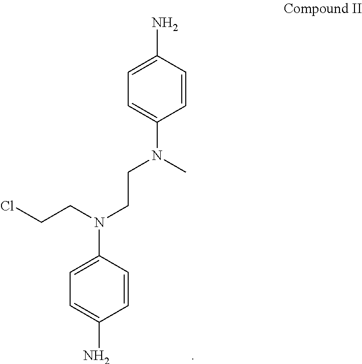 [SMILES: CN(CCN(CCCl)c1ccc(N)cc1)c1ccc(N)cc1]

98. A method for screening a potential cancer therapeutic, comprising the steps of:
growing cells containing a chromosomal translocation of the Mixed Lineage Leukemia (Mll) gene in the presence of a compound suspected of being a cancer therapeutic, growing said cells in the presence of a reference compound, determining the rate of growth of said cells in the presence of said compound and the rate of growth of said cells in the presence of a reference compound and comparing the growth rate of said cells, wherein a slower rate of growth of said cells in the presence of said compound than in the presence of said reference compound is indicative of a cancer therapeutic, wherein said reference compound is a compound of Compound III or a pharmaceutically acceptable salt, solvate, hydrate or stereoisomer thereof:

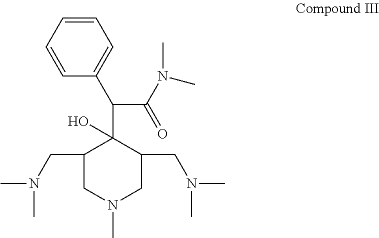

99. A method for screening a potential cancer therapeutic, comprising the steps of: growing cells containing a chromosomal translocation of the Mixed Lineage Leukemia (Mll) gene in the presence of a compound suspected of being a cancer therapeutic, growing said cells in the presence of a reference compound, determining the rate of growth of said cells in the presence of said compound and the rate of growth of said cells in the presence of a reference compound and comparing the growth rate of said cells, wherein a slower rate of growth of said cells in the presence of said compound than in the presence of said reference compound is indicative of a cancer therapeutic, wherein said reference compound is a compound of Compound IV or a pharmaceutically acceptable salt, solvate, hydrate or stereoisomer thereof:

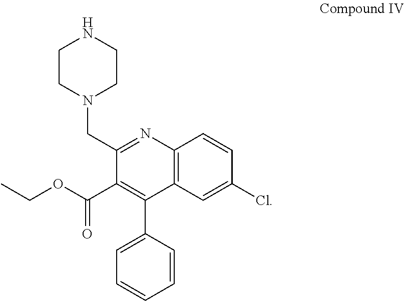 [SMILES: CCOC(=O)c1c(CN2CCNCC2)nc2ccc(Cl)cc2c1-c1ccccc1]

100. The method according to any one of claims 95-99, wherein said cells are human cells.

101. The method according to any one of claims 95-99, wherein said cells are an MLL-AF9 transformed cell line.

102. A method of screening a potential diabetes therapeutic comprising the steps of: growing pancreatic β-cells in the presence of a compound suspected of being a diabetes therapeutic, growing pancreatic β-cells in the presence of a reference compound, determining the rate of growth of the pancreatic β-cells in the presence of said compound and the rate of growth of the pancreatic β-cells in the presence of the reference compound and comparing the growth rate of the pancreatic β-cells, wherein a higher rate of growth of pancreatic β-cells in the presence of said compound than in the presence of said reference compound is indicative of a diabetes therapeutic, wherein said reference compound is a compound of Formula B or a pharmaceutically acceptable salt, solvate, hydrate or stereoisomer thereof.

103. A methods of identifying a compound that enhances pancreatic β-cell proliferation, the methods comprising the steps of: growing β-cells in the presence of a compound suspected of enhancing pancreatic β-cell proliferation, growing pancreatic β-cells in the presence of a reference compound, determining the rate of growth of the pancreatic β-cells in the presence of said compound and the rate of growth of the pancreatic β-cells in the presence of the reference compound and comparing the growth rate of the pancreatic β-cells, wherein a higher rate of growth of pancreatic β-cells in the presence of said compound than in the presence of said reference compound is indicative of a compound that enhances pancreatic β-cell proliferation, wherein said reference compound is a compound of Formula B or a pharmaceutically acceptable salt, solvate
